(19) United States
(12) Patent Application Publication
(10) Pub. No.: US 2025/0029016 A1
(43) Pub. Date: Jan. 23, 2025

(54) LINEAR MACHINE LEARNING METHOD BASED ON DNA HYBRIDIZATION REACTION TECHNOLOGY

(71) Applicant: Yanshan University, Qinhuangdao (CN)

(72) Inventors: Chengye Zou, Qinhuangdao (CN); Haifeng Li, Qinhuangdao (CN); Wenlong Wang, Qinhuangdao (CN)

(21) Appl. No.: 18/753,150

(22) Filed: Jun. 25, 2024

(30) Foreign Application Priority Data
Jul. 3, 2023 (CN) 2023108023956

(51) Int. Cl. G06N 99/00

(52) U.S. Cl. CPC G06N 99/007

(57) ABSTRACT

A new linear machine learning method based on DNA hybridization reaction technology, includes a machine learning training part, an algorithm part, and a testing part. This machine learning method has the ability to learn linear functions. Unlike silicon circuits, the learning algorithm is implemented through the synchronization of DNA hybridization reactions. Therefore, the calculation mode of this machine learning method is a parallel computing model, and the weights of this machine learning are obtained through training without the involvement of electronic computers. Through the method, it is possible to learn multivariable linear functions without any limitation on the number of input terms. Due to the non-negative DNA concentration, the method used a dual track model for negative data processing operations.

### TECHNICAL FIELD

The disclosure relates to the fields of biocomputing and machine learning, and particularly to a linear machine learning method based on deoxyribonucleic acid (DNA) hybridization reaction technology.

### BACKGROUND

Since the invention of electronic computer in 1946, computer technology has penetrated into every aspect of people's lives and work, making a huge contribution to the development of society. Due to the rapid development of science and technology in recent years, the calculation time for solving technological problems using traditional electronic computers has increased exponentially with the increase of problem-solving scale. In order to meet the growing demand for large-scale and ultra-large-scale computing, the characteristics of high-performance computing and accelerated computing performance are needed. Breaking through the constraints of silicon semiconductor devices and developing non-traditional computers are important ways of future computing technology. DNA computing based on DNA molecules includes DNA computing models based on DNA hybridization reactions, DNA computing models based on DNA enzyme, DNA computing models based on DNA tile, and DNA computing models based on nanoparticle. DNA hybridization technology is an important research method in the DNA computing. The driving force of DNA hybridization comes from the intermolecular forces under the condition of complementary Waston Crick bases. The DNA hybridization reaction process has features of parallelism, programmability, autonomy, dynamic cascading, high information storage, and low power consumption, and spontaneously occurs at room temperature. By utilizing single-molecule self-assembly and fluorescence labeling, the DNA hybridization technology has been applied in biosensors, molecular detection DNA nanorobots, drug delivery and diagnosis and treatment. In addition, the DNA hybridization technology can combine with nanoparticles, quantum dots, and proteins to promote the development of parallel computing models, integrated cryptography, and nanoelectronics.

Learning ability is an important indicator of human intelligence, and the purpose of machine learning is to enable machines to have learning ability. The machine learning includes supervised learning (SL), unsupervised learning (UL), semi-supervised learning (SSL), deep learning (DL), reinforcement learning (RL), and transfer learning (TR). Methods of the machine learning include mechanical learning, inductive learning, explanation-based learning, genetic-algorithm-based learning, and neural-network-based learning. By utilizing the controllability and the ability to be compiled of DNA molecules, various algorithms of machine learning are implemented to achieve a part of functions of the human brain.

At present, the learning process of most artificial intelligence systems based on DNA molecular computing requires the assistance of electronic computers. The weight update in artificial intelligence systems is not completed by DNA molecular circuits, but by the assistance of electronic computing or existing databases (such as large handwritten digit databases). The weight update process is the learning and training process of artificial intelligence systems, and it is also the core content of artificial intelligence systems. Therefore, most artificial intelligence systems based on DNA molecular computing currently do not have true learning ability, only have certain recognition and classification functions.

Given the above, the present disclosure provides a new linear machine learning method based on DNA hybridization reaction technology.

### SUMMARY

At present, the learning process of most artificial intelligence systems based on DNA molecular computing requires the assistance of electronic computers. In order to overcome the shortcomings of the prior art, the present disclosure provides a new linear machine learning method based on DNA hybridization reaction technology (also referred to as linear machine learning method based on DNA hybridization reaction technology). A machine learning system based on DNA molecular circuits provided by the present disclosure can complete the training and testing process without the assistance of electronic computers, achieving complete biological machine learning.

The system includes three parts, including a machine learning training part, an algorithm part, and a testing part. The linear machine learning method based on DNA hybridization reaction technology has the ability to learn linear functions. Unlike silicon circuits, the learning algorithm is implemented through the synchronization of DNA hybridization reactions. Therefore, the computing mode of the machine learning method is a parallel computing model, and the weights of this machine learning are obtained through training without the involvement of electronic computers. Through the method, it is possible to learn multivariable linear functions without any limitation on the number of input terms. Due to the non-negative DNA concentration, the method uses a dual track model for negative data processing operations.

The linear machine learning method based on DNA hybridization reaction technology, includes: a training part, an algorithm part and a testing part.

(1) Reaction expressions of the training part are as follows:

$${X}_{1}^{+} + {W}_{1}^{+} {⟶}^{{k}_{1}} {I}^{+} + {X}_{1}^{+} + {W}_{1}^{+} \qquad ( 1 a )$$

$${X}_{1}^{-} + {W}_{1}^{-} {⟶}^{{k}_{1}} {I}^{+} + {X}_{1}^{-} + {W}_{1}^{-} \qquad ( 1 b )$$

$${X}_{1}^{+} + {W}_{1}^{-} {⟶}^{{k}_{1}} {I}^{-} + {X}_{1}^{+} + {W}_{1}^{-} \qquad ( 1 c )$$

$${X}_{1}^{-} + {W}_{1}^{+} {⟶}^{{k}_{1}} {I}^{-} + {X}_{1}^{-} + {W}_{1}^{+} \qquad ( 1 d )$$

$${X}_{2}^{+} + {W}_{2}^{+} {⟶}^{{k}_{1}} {I}^{+} + {X}_{2}^{+} + {W}_{2}^{+} \qquad ( 2 a )$$

$${X}_{2}^{-} + {W}_{2}^{-} {⟶}^{{k}_{1}} {I}^{+} + {X}_{2}^{-} + {W}_{2}^{-} \qquad ( 2 b )$$

$${X}_{2}^{+} + {W}_{2}^{-} {⟶}^{{k}_{1}} {I}^{-} + {X}_{2}^{+} + {W}_{2}^{-} \qquad ( 2 c )$$

$${X}_{2}^{-} + {W}_{2}^{+} {⟶}^{{k}_{1}} {I}^{-} + {X}_{2}^{-} + {W}_{2}^{+} \qquad ( 2 d )$$

$$⋮$$

$${X}_{N}^{+} + {W}_{N}^{+} {⟶}^{{k}_{1}} {I}^{+} + {X}_{N}^{+} + {W}_{N}^{+} \qquad ( 3 a )$$

$${X}_{N}^{-} + {W}_{N}^{-} {⟶}^{{k}_{1}} {I}^{+} + {X}_{N}^{-} + {W}_{N}^{-} \qquad ( 3 d )$$

$${X}_{N}^{+} + {W}_{N}^{-} {⟶}^{{k}_{1}} {I}^{-} + {X}_{N}^{+} + {W}_{N}^{-} \qquad ( 3 c )$$

$${X}_{N}^{-} + {W}_{N}^{+} {⟶}^{{k}_{1}} {I}^{-} + {X}_{N}^{-} + {W}_{N}^{+} \qquad ( 3 d )$$

$${Y}^{+} + {H}^{+} {⟶}^{{k}_{1}} {I}^{-} + {H}^{+} \qquad ( 4 a )$$

$${Y}^{-} + {H}^{-} {⟶}^{{k}_{1}} {I}^{+} + {H}^{-} \qquad ( 4 b )$$

$${I}^{+} + {C}^{+} {⟶}^{{k}_{1}} {I}^{+} + {C}^{+} + {Y}^{+} \qquad ( 4 c )$$

$${I}^{-} + {C}^{+} {⟶}^{{k}_{1}} {I}^{-} + {C}^{-} + {Y}^{+} \qquad ( 4 d )$$

$${X}_{1}^{+} + {X}_{1}^{-} {⟶}^{{k}_{2}} Φ \qquad ( 5 a )$$

$${W}_{1}^{+} + {W}_{1}^{-} {⟶}^{{k}_{2}} Φ \qquad ( 5 b )$$

$${X}_{2}^{+} + {X}_{2}^{-} {⟶}^{{k}_{2}} Φ \qquad ( 5 c )$$

$${W}_{2}^{+} + {W}_{2}^{-} {⟶}^{{k}_{2}} Φ \qquad ( 5 d )$$

$$⋮$$

$${X}_{i}^{+} + {X}_{i}^{-} {⟶}^{{k}_{2}} Φ \qquad ( 6 a )$$

$${W}_{i}^{+} + {W}_{i}^{-} {⟶}^{{k}_{2}} Φ \qquad ( 6 b )$$

$${Y}^{+} + {Y}^{-} {⟶}^{{k}_{2}} Φ \qquad ( 6 c )$$

$${C}^{+} + {C}^{-} {⟶}^{{k}_{2}} Φ \qquad ( 6 d )$$

$${H}^{+} + {H}^{-} {⟶}^{{k}_{2}} Φ \qquad ( 6 e )$$

$${I}^{+} + {I}^{-} {⟶}^{{k}_{2}} Φ \qquad ( 6 f )$$

The reactions (1a) to (3d) belong to a catalytic reaction module 1 (also referred to as first catalytic reaction module). The reaction (4a) and the reaction (4b) belong to a catalytic reaction module 2 (also referred to as second catalytic reaction module). The reaction (4c) and the reaction (4d) belong to the catalytic reaction module 1 and reactions (5a) to (6f) belong to an annihilation reaction.

Where [Xᵢ]ₜ=[Wᵢ⁺]ₜ−[Xᵢ⁻]ₜ, i=1, 2 . . . , N, N represents the number of input items, [*]ₜ represents a concentration of a substance * at a time t, and x, represents an i-th input value. [Wᵢ]ₜ=[Wᵢ⁺]ₜ−[Wᵢ⁻]ₜ, Wᵢ represents an i-th weight, and differential equations of concentrations varying over time of substances I⁺ and I⁻ are:

$$\{ \begin{matrix}\frac{d [ {I}^{+} ]}{dt} = {k}_{1} ( {{[ {W}_{1}^{+} ]}_{t} [ {X}_{1}^{+} ]}_{t} + {{[ {W}_{2}^{+} ]}_{t} [ {X}_{2}^{+} ]}_{t} + … + \\ {{[ {W}_{N}^{+} ]}_{t} [ {X}_{N}^{+} ]}_{t} + {{[ {W}_{1}^{-} ]}_{t} [ {X}_{1}^{-} ]}_{t} + {{[ {W}_{2}^{-} ]}_{t} [ {X}_{2}^{-} ]}_{t} + … + \\ {{[ {W}_{N}^{-} ]}_{t} [ {X}_{N}^{-} ]}_{t} + {{[ {Y}^{-} ]}_{t} [ {H}^{-} ]}_{t} ) \\ \frac{d [ {I}^{-} ]}{dt} = {k}_{1} ( {{[ {W}_{1}^{-} ]}_{t} [ {X}_{1}^{-} ]}_{t} + {{[ {W}_{2}^{+} ]}_{t} [ {X}_{2}^{-} ]}_{t} + … + \\ {{[ {W}_{N}^{+} ]}_{t} [ {X}_{N}^{-} ]}_{t} + {{[ {W}_{1}^{-} ]}_{t} [ {X}_{1}^{+} ]}_{t} + {{[ {W}_{2}^{-} ]}_{t} [ {X}_{2}^{+} ]}_{t} + … + \\ {{[ {W}_{N}^{-} ]}_{t} [ {X}_{N}^{+} ]}_{t} + {{[ {Y}^{+} ]}_{t} [ {H}^{+} ]}_{t} )\end{matrix} \qquad ( 7 )$$

The differential equations (7) are simplified as follows:

$$\{ \begin{matrix}\frac{{d [ {I}^{+} ]}_{t}}{dt} = {k}_{1} ( {\sum }_{i = 1}^{N} ( {{[ {W}_{i}^{+} ]}_{t} [ {X}_{i}^{+} ]}_{t} + {{[ {W}_{i}^{-} ]}_{t} [ {X}_{i}^{-} ]}_{t} ) + {{[ {Y}^{-} ]}_{t} [ {H}^{-} ]}_{t} ) \\ \frac{{d [ {I}^{-} ]}_{t}}{dt} = {k}_{1} ( {\sum }_{i = 1}^{N} ( {{[ {W}_{i}^{+} ]}_{t} [ {X}_{i}^{-} ]}_{t} + {{[ {W}_{i}^{-} ]}_{t} [ {X}_{i}^{+} ]}_{t} ) + {{[ {Y}^{+} ]}_{t} [ {H}^{+} ]}_{t} )\end{matrix}$$

And:

$$\begin{matrix}\frac{{d [ I ]}_{t}}{d t} = \frac{{d [ {I}^{+} ]}_{t}}{d t} - \frac{{d [ {I}^{-} ]}_{t}}{d t} \\ = \begin{matrix}{k}_{1} ( {\sum }_{i = 1}^{N} ( {{[ {W}_{i}^{+} ]}_{t} [ {X}_{i}^{+} ]}_{t} + {{[ {W}_{i}^{-} ]}_{t} [ {X}_{i}^{-} ]}_{t} - {{[ {W}_{i}^{+} ]}_{t} [ {X}_{i}^{-} ]}_{t} - \\ {{[ {W}_{i}^{-} ]}_{t} [ {X}_{i}^{+} ]}_{t} + {{[ {Y}^{-} ]}_{t} [ {H}^{-} ]}_{t} - {{[ {Y}^{+} ]}_{t} [ {H}^{+} ]}_{t} ) )\end{matrix}\end{matrix}$$

Supposing [H⁺]ₜ=[H⁻]ₜ=1 nanomoles per liter (nM), and

$$\frac{d I ( t )}{d t} = {k}_{1} ( {\sum }_{i = 1}^{N} {W}_{i} ( t ) {X}_{i} ( t ) - Y ( t ) ) ,$$

when a DNA hybridization reaction network reaches dynamic equilibrium, i.e.,

$$\frac{d I ( t )}{d t} = 0 ,$$

then:

$$Y = {\sum }_{i = 1}^{N} {W}_{i} {X}_{i}$$

where Y(t)=[Y⁺]ₜ−[Y⁻]ₜ, Y represents an output value of a system.

(2) Reaction expressions of the algorithm part are as follows:

$${D}^{+} + {X}_{1}^{+} {\to }^{{k}_{3}} {D}^{+} + {X}_{1}^{+} + {W}_{1}^{+} \qquad ( 8 a )$$

$${D}^{-} + {X}_{1}^{-} {\to }^{{k}_{3}} {D}^{-} + {X}_{1}^{-} + {W}_{1}^{+} \qquad ( 8 b )$$

$${Y}^{+} + {X}_{1}^{-} {\to }^{{k}_{3}} {Y}^{+} + {X}_{1}^{-} + {W}_{1}^{+} \qquad ( 8 c )$$

$${Y}^{-} + {X}_{1}^{+} {\to }^{{k}_{3}} {D}^{-} + {X}_{1}^{+} + {W}_{1}^{+} \qquad ( 8 d )$$

$${D}^{+} + {X}_{1}^{-} {\to }^{{k}_{3}} {D}^{+} + {X}_{1}^{-} + {W}_{1}^{-} \qquad ( 8 e )$$

$${D}^{-} + {X}_{1}^{+} {\to }^{{k}_{3}} {D}^{-} + {X}_{1}^{+} + {W}_{1}^{-} \qquad ( 8 f )$$

$${Y}^{+} + {X}_{1}^{+} {\to }^{{k}_{3}} {Y}^{+} + {X}_{1}^{+} + {W}_{1}^{-} \qquad ( 8 g )$$

$${Y}^{-} + {X}_{1}^{-} {\to }^{{k}_{3}} D + {X}_{1}^{-} + {W}_{1}^{-} \qquad ( 8 h )$$

$${D}^{+} + {X}_{2}^{+} {\to }^{{k}_{3}} {D}^{+} + {X}_{2}^{+} + {W}_{2}^{+} \qquad ( 9 a )$$

$${D}^{-} + {X}_{2}^{-} {\to }^{{k}_{3}} {D}^{-} + {X}_{2}^{-} + {W}_{2}^{+} \qquad ( 9 b )$$

$${Y}^{+} + {X}_{2}^{-} {\to }^{{k}_{3}} {Y}^{+} + {X}_{2}^{-} + {W}_{2}^{+} \qquad ( 9 c )$$

$${Y}^{-} + {X}_{2}^{+} {\to }^{{k}_{3}} {D}^{-} + {X}_{2}^{+} + {W}_{2}^{+} \qquad ( 9 d )$$

$${D}^{+} + {X}_{2}^{-} {\to }^{{k}_{3}} {D}^{+} + {X}_{2}^{-} + {W}_{2}^{-} \qquad ( 9 e )$$

$${D}^{-} + {X}_{2}^{+} {\to }^{{k}_{3}} {D}^{-} + {X}_{2}^{+} + {W}_{2}^{-} \qquad ( 9 f )$$

$${Y}^{+} + {X}_{2}^{+} {\to }^{{k}_{3}} {Y}^{+} + {X}_{2}^{+} + {W}_{2}^{-} \qquad ( 9 g )$$

$${Y}^{-} + {X}_{2}^{-} {\to }^{{k}_{3}} {D}^{-} + {X}_{2}^{-} + {W}_{2}^{-} \qquad ( 9 h )$$

$$⋮$$

$${D}^{+} + {X}_{N}^{+} {\to }^{{k}_{3}} {D}^{+} + {X}_{N}^{+} + {W}_{N}^{+} \qquad ( 10 a )$$

$${D}^{-} + {X}_{N}^{-} {\to }^{{k}_{3}} {D}^{-} + {X}_{N}^{-} + {W}_{N}^{+} \qquad ( 10 b )$$

$${Y}^{+} + {X}_{N}^{-} {\to }^{{k}_{3}} {Y}^{+} + {X}_{N}^{-} + {W}_{N}^{+} \qquad ( 10 c )$$

$${Y}^{-} + {X}_{N}^{+} {\to }^{{k}_{3}} {D}^{-} + {X}_{N}^{+} + {W}_{N}^{+} \qquad ( 10 d )$$

$${D}^{+} + {X}_{N}^{-} {\to }^{{k}_{3}} {D}^{+} + {X}_{N}^{-} + {W}_{N}^{-} \qquad ( 10 e )$$

$${D}^{-} + {X}_{N}^{+} {\to }^{{k}_{3}} {D}^{-} + {X}_{N}^{+} + {W}_{N}^{-} \qquad ( 10 f )$$

$${Y}^{+} + {X}_{N}^{+} {\to }^{{k}_{3}} {Y}^{+} + {X}_{N}^{+} + {W}_{N}^{-} \qquad ( 10 g )$$

$${Y}^{-} + {X}_{N}^{+} {\to }^{{k}_{3}} {D}^{-} + {X}_{N}^{-} + {W}_{N}^{-} \qquad ( 10 h )$$

Where reactions (8)-(10) belong to the catalytic reaction module 1.

$$\{ \begin{matrix}\frac{d [ {W}_{1}^{+} ]}{d t} = {k}_{3} ( {{[ {D}^{+} ]}_{t} [ {X}_{1}^{+} ]}_{t} + {{[ {D}^{-} ]}_{t} [ {X}_{1}^{-} ]}_{t} + {{[ {Y}^{+} ]}_{t} [ {X}_{1}^{-} ]}_{t} + {{[ {Y}^{-} ]}_{t} [ {X}_{1}^{+} ]}_{t} ) \\ \frac{d [ {W}_{1}^{-} ]}{d t} = {k}_{3} ( {{[ {D}^{+} ]}_{t} [ {X}_{1}^{-} ]}_{t} + {{[ {D}^{-} ]}_{t} [ {X}_{1}^{+} ]}_{t} + {{[ {Y}^{+} ]}_{t} [ {X}_{1}^{+} ]}_{t} + {{[ {Y}^{-} ]}_{t} [ {X}_{1}^{-} ]}_{t} )\end{matrix} \qquad ( 11 )$$

$$\{ \begin{matrix}\frac{d [ {W}_{2}^{+} ]}{d t} = {k}_{3} ( {{[ {D}^{+} ]}_{t} [ {X}_{2}^{+} ]}_{t} + {{[ {D}^{-} ]}_{t} [ {X}_{2}^{-} ]}_{t} + {{[ {Y}^{+} ]}_{t} [ {X}_{2}^{-} ]}_{t} + {{[ {Y}^{-} ]}_{t} [ {X}_{2}^{+} ]}_{t} ) \\ \frac{d [ {W}_{2}^{-} ]}{d t} = {k}_{3} ( {{[ {D}^{+} ]}_{t} [ {X}_{2}^{-} ]}_{t} + {{[ {D}^{-} ]}_{t} [ {X}_{2}^{+} ]}_{t} + {{[ {Y}^{+} ]}_{t} [ {X}_{2}^{+} ]}_{t} + {{[ {Y}^{-} ]}_{t} [ {X}_{2}^{-} ]}_{t} )\end{matrix} \qquad ( 12 )$$

$$⋮$$

$$\{ \begin{matrix}\frac{d [ {W}_{N}^{+} ]}{d t} = {k}_{3} ( {{[ {D}^{+} ]}_{t} [ {X}_{N}^{+} ]}_{t} + {{[ {D}^{-} ]}_{t} [ {X}_{N}^{-} ]}_{t} + {{[ {Y}^{+} ]}_{t} [ {X}_{N}^{-} ]}_{t} + {{[ {Y}^{-} ]}_{t} [ {X}_{N}^{+} ]}_{t} ) \\ \frac{d [ {W}_{N}^{-} ]}{d t} = {k}_{3} ( {{[ {D}^{+} ]}_{t} [ {X}_{N}^{-} ]}_{t} + {{[ {D}^{-} ]}_{t} [ {X}_{N}^{+} ]}_{t} + {{[ {Y}^{+} ]}_{t} [ {X}_{N}^{+} ]}_{t} + {{[ {Y}^{-} ]}_{t} [ {X}_{N}^{-} ]}_{t} )\end{matrix} \qquad ( 13 )$$

where D(t)=[D⁺]ₜ−[D⁻]ₜ, D represents an expectation value.

Concluding from differential equations (11)-(13), what is obtained is:

$$\begin{matrix}\frac{d {W}_{1} ( t )}{d t} = \frac{{d [ {W}_{1}^{+} ]}_{t}}{d t} - \frac{{d [ {W}_{1}^{-} ]}_{t}}{d t} \\ = \begin{matrix}{k}_{3} ( {{[ {D}^{+} ]}_{t} [ {X}_{1}^{+} ]}_{t} + {{[ {D}^{-} ]}_{t} [ {X}_{1}^{-} ]}_{t} - {{[ {D}^{+} ]}_{t} [ {X}_{1}^{-} ]}_{t} - {{[ D ]}_{t} [ {X}_{1}^{+} ]}_{t} ) + \\ {k}_{2} ( {{[ {Y}^{+} ]}_{t} [ {X}_{1}^{-} ]}_{t} + {{[ {Y}^{-} ]}_{t} [ {X}_{1}^{+} ]}_{t} - {{[ {Y}^{+} ]}_{t} [ {X}_{1}^{+} ]}_{t} + {{[ {Y}^{-} ]}_{t} [ {X}_{1}^{-} ]}_{t} )\end{matrix} \\ = {k}_{3} [ ( {[ {D}^{+} ]}_{t} - {[ {D}^{-} ]}_{t} ) - ( {[ {Y}^{+} ]}_{t} - {[ {Y}^{-} ]}_{t} ) ] \times ( {[ {X}_{1}^{+} ]}_{t} - {[ {X}_{1}^{-} ]}_{t} ) \\ = {{k}_{3} ( {[ D ]}_{t} - {[ Y ]}_{t} ) [ {X}_{1} ]}_{t}\end{matrix}$$

$$\begin{matrix}\frac{d {W}_{2} ( t )}{d t} = \frac{{d [ {W}_{2}^{+} ]}_{t}}{d t} - \frac{{d [ {W}_{2}^{-} ]}_{t}}{d t} \\ = \begin{matrix}{k}_{3} ( {{[ {D}^{+} ]}_{t} [ {X}_{2}^{+} ]}_{t} + {{[ {D}^{-} ]}_{t} [ {X}_{2}^{-} ]}_{t} - {{[ {D}^{+} ]}_{t} [ {X}_{2}^{-} ]}_{t} - {{[ {D}^{-} ]}_{t} [ {X}_{2}^{*} ]}_{t} ) + \\ {k}_{2} ( {{[ {Y}^{+} ]}_{t} [ {X}_{2}^{-} ]}_{t} + {{[ {Y}^{-} ]}_{t} [ {X}_{2}^{+} ]}_{t} - {{[ {Y}^{+} ]}_{t} [ {X}_{2}^{+} ]}_{t} + {{[ {Y}^{-} ]}_{t} [ {X}_{2}^{-} ]}_{t}\end{matrix} \\ = {k}_{3} [ ( {[ {D}^{+} ]}_{t} - {[ {D}^{-} ]}_{t} ) - ( {[ {Y}^{+} ]}_{t} - {[ {Y}^{-} ]}_{t} ) ] \times ( {[ {X}_{2}^{+} ]}_{t} - {[ {X}_{2}^{-} ]}_{t} ) \\ = {{k}_{3} ( {[ D ]}_{t} - {[ Y ]}_{t} ) [ {X}_{2} ]}_{t}\end{matrix}$$

$$⋮$$

$$\begin{matrix}\frac{d {W}_{N} ( t )}{d t} = \frac{{d [ {W}_{N}^{+} ]}_{t}}{d t} - \frac{{d [ {W}_{N}^{-} ]}_{t}}{d t} \\ = \begin{matrix}{k}_{3} ( {{[ {D}^{+} ]}_{t} [ {X}_{N}^{+} ]}_{t} + {{[ {D}^{-} ]}_{t} [ {X}_{N}^{-} ]}_{t} - {{[ {D}^{+} ]}_{t} [ {X}_{N}^{-} ]}_{t} - \\ {{[ {D}^{-} ]}_{t} [ {X}_{N}^{*} ]}_{t} ) + {k}_{2} ( {{[ {Y}^{+} ]}_{t} [ {X}_{N}^{-} ]}_{t} + {{[ {Y}^{-} ]}_{t} [ {X}_{N}^{*} ]}_{t}\end{matrix} \\ = {k}_{3} [ ( {[ {D}^{+} ]}_{t} - {[ {D}^{-} ]}_{t} ) - ( {[ {Y}^{+} ]}_{t} - {[ {Y}^{-} ]}_{t} ) ] \times ( {[ {X}_{N}^{+} ]}_{t} - {[ {X}_{N}^{-} ]}_{t} ) \\ = {{k}_{3} ( {[ D ]}_{t} - {[ Y ]}_{t} ) [ {X}_{N} ]}_{t} .\end{matrix}$$

(3) Reaction expressions of the testing part are as follows:

$${X}_{1}^{+} + {\hat{W}}_{1}^{+} {\to }^{{k}_{1}} {\hat{I}}^{+} + {X}_{1}^{+} + {\hat{W}}_{1}^{+} \qquad ( 14 a )$$

$${X}_{1}^{-} + {\hat{W}}_{1}^{-} {\to }^{{k}_{1}} {\hat{I}}^{+} + {X}_{1}^{-} + {\hat{W}}_{1}^{+} \qquad ( 14 b )$$

$${X}_{1}^{+} + {\hat{W}}_{1}^{-} {\to }^{{k}_{1}} {\hat{I}}^{-} + {X}_{1}^{+} + {\hat{W}}_{1}^{-} \qquad ( 14 c )$$

$${X}_{1}^{-} + {\hat{W}}_{1}^{+} {\to }^{{k}_{1}} {\hat{I}}^{-} + {X}_{1}^{-} + {\hat{W}}_{1}^{+} \qquad ( 14 d )$$

$${X}_{2}^{+} + {\hat{W}}_{2}^{+} {\to }^{{k}_{1}} {\hat{I}}^{+} + {X}_{2}^{+} + {\hat{W}}_{2}^{+} \qquad ( 15 a )$$

$${X}_{2}^{+} + {\hat{W}}_{2}^{+} {\to }^{{k}_{1}} {\hat{I}}^{+} + {X}_{2}^{+} + {\hat{W}}_{2}^{+} \qquad ( 15 b )$$

$${X}_{2}^{+} + {\hat{W}}_{2}^{-} {\to }^{{k}_{1}} {\hat{I}}^{-} + {X}_{2}^{+} + {\hat{W}}_{2}^{-} \qquad ( 15 c )$$

$${X}_{2}^{-} + {\hat{W}}_{2}^{+} {\to }^{{k}_{1}} {\hat{I}}^{-} + {X}_{2}^{-} + {\hat{W}}_{2}^{+} \qquad ( 15 d )$$

$$⋮$$

$${X}_{N}^{+} + {\hat{W}}_{N}^{+} {\to }^{{k}_{1}} {\hat{I}}^{+} + {X}_{N}^{+} + {\hat{W}}_{N}^{+} \qquad ( 16 a )$$

$${X}_{N}^{-} + {\hat{W}}_{N}^{-} {\to }^{{k}_{1}} {\hat{I}}^{+} + {X}_{N}^{-} + {\hat{W}}_{N}^{-} \qquad ( 16 b )$$

$${X}_{N}^{+} + {\hat{W}}_{N}^{-} {\to }^{{k}_{1}} {\hat{I}}^{-} + {X}_{N}^{+} + {\hat{W}}_{N} \qquad ( 16 c )$$

$${X}_{N}^{-} + {\hat{W}}_{N}^{+} {\to }^{{k}_{1}} {\hat{I}}^{-} + {X}_{N}^{-} + {\hat{W}}_{N}^{+} \qquad ( 16 d )$$

$${\hat{Y}}^{+} + {\hat{H}}^{+} {\to }^{{k}_{1}} {\hat{I}}^{-} + {\hat{H}}^{+} \qquad ( 17 a )$$

$${\hat{Y}}^{-} + {\hat{H}}^{-} {\to }^{{k}_{1}} {\hat{I}}^{+} + {\hat{H}}^{-} \qquad ( 17 b )$$

$${\hat{I}}^{+} + {\hat{C}}^{+} {\to }^{{k}_{1}} {\hat{I}}^{+} + {\hat{C}}^{+} + {\hat{Y}}^{+} \qquad ( 17 c )$$

$${\hat{I}}^{-} + {\hat{C}}^{-} {\to }^{{k}_{1}} {\hat{I}}^{-} + {\hat{C}}^{-} + {\hat{Y}}^{+} \qquad ( 17 d )$$

$${X}_{i}^{+} + {X}_{i}^{-} {\to }^{{k}_{2}} Φ \qquad ( 18 a )$$

$${\hat{W}}_{i}^{+} + \hat{{W}_{i}^{-}} {\to }^{{k}_{2}} Φ \qquad ( 18 b )$$

$${\hat{Y}}^{+} + \hat{{Y}^{-}} {\to }^{{k}_{2}} Φ \qquad ( 18 c )$$

$${\hat{C}}^{+} + \hat{{C}^{-}} {\to }^{{k}_{2}} Φ \qquad ( 18 d )$$

$${\hat{H}}^{+} + \hat{{H}^{-}} {\to }^{{k}_{2}} Φ \qquad ( 18 e )$$

$${\hat{I}}^{+} + \hat{{I}^{-}} {\to }^{{k}_{2}} Φ \qquad ( 18 f )$$

where reactions (14d) to (16d) belong to the catalytic reaction module 1. Reactions (17a) and (17b) belong to the catalytic reaction module 2; reactions (17c) and (17d) belong to the first catalytic module and reactions (18a)-(18f) belong to the annihilation reaction module.

The beneficial effects of above technical solutions are as follows.

(1) The machine learning system based on DNA molecular circuits provided by the present disclosure has the ability to autonomously learn and can complete the training and testing process without the assistance of electronic computers (the weights of the machine learning are obtained through training), achieving complete biological machine learning without the involvement of electronic computers.

(2) The multivariate linear function relationship is f(x₁, x₂, . . . , x_{N})=w₁x₁+w₂x₂+ . . . +w_{N}x_{N}. The number of independent variables is N, and artificial neural networks of existing research results based on DNA molecule computing can only handle the linear function relationship of two independent variables. Through the method, multivariate linear functions can be learned without any limit on the number of input items, which can be any positive integer.

(3) Due to the fact that the input and output values of DNA molecular circuits are represented by the concentration of DNA molecules, which is non-negative, data with negative numbers cannot be processed. The present disclosure adopts a bimolecular concentration difference to represent the input and output values, as the concentration difference can be positive or negative and can represent all real numbers.

(4) The present disclosure can be used for fitting and predicting the relationship between total current and voltage in parallel circuits, and can also predict the value of partial resistance.

### BRIEF DESCRIPTION OF DRAWINGS

FIG. 1 illustrates a flowchart of the present disclosure.

FIG. 2 illustrates a schematic diagram of main DNA strands displacement reaction of a first submodule of a catalytic reaction module 1 of the present disclosure.

FIG. 3 illustrates a schematic diagram of main DNA strands displacement reaction of a second submodule of the catalytic reaction module 1 of the present disclosure.

FIG. 4 illustrates a schematic diagram of main DNA strands displacement reaction of a third submodule of the catalytic reaction module 1 of the present disclosure.

FIG. 5 illustrates a schematic diagram of main DNA strands displacement reaction of a fourth submodule of the catalytic reaction module 1 of the present disclosure.

FIG. 6 illustrates a schematic diagram of main DNA strands displacement reaction of a catalytic reaction module 2 of the present disclosure.

FIG. 7 illustrates a schematic diagram of main DNA strands displacement reaction of an annihilation reaction module of the present disclosure.

FIG. 8 illustrated a circuit diagram of a parallel circuit of the present disclosure.

FIG. 9 illustrated a schematic diagram of weight update trajectory of the present disclosure.

FIG. 10 illustrated a schematic diagram of evolution of average relative error with training times of the present disclosure.

FIG. 11 illustrated a schematic diagram of variation of training times with the number of training rounds of the present disclosure.

FIG. 12 illustrated a schematic diagram of evolution of relative error with test data of the present disclosure.

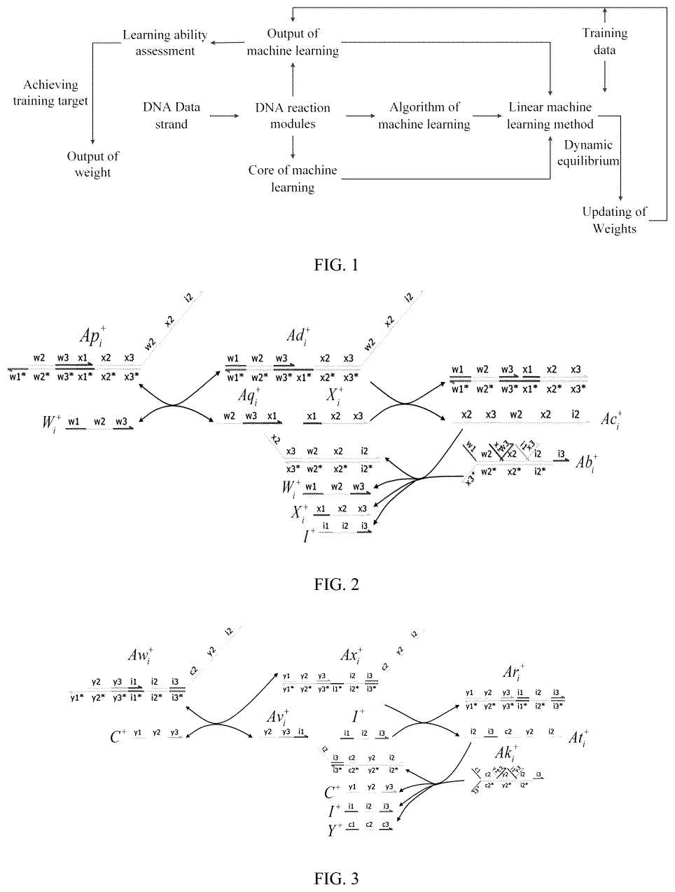

### DETAILED DESCRIPTION OF EMBODIMENTS

In order to clarify the purpose, technical solutions, and advantages of the embodiments of the present disclosure, a clear and complete description of the technical solutions in the embodiments of the present disclosure will be provided below. Apparently, the described embodiments are a part of the embodiments of the present disclosure, not all embodiments. The following is a detailed description of the present disclosure in conjunction with the accompanying drawings. However, it should be understood that the provision of the drawings is only for a better understanding of the present disclosure, and they should not be understood as limitations to the present disclosure.

Specific steps are as follows.

### 1. Design of Linear Machine Learning Based on Idealized Reaction.

(1) The training part of machine learning:

$${X}_{1}^{+} + {W}_{1}^{+} {\to }^{{k}_{1}} {I}^{+} + {X}_{1}^{+} + {W}_{1}^{+} \qquad ( 1 a )$$

$${X}_{1}^{-} + {W}_{1}^{-} {\to }^{{k}_{1}} {I}^{+} + {X}_{1}^{-} + {W}_{1}^{-} \qquad ( 1 b )$$

$${X}_{1}^{+} + {W}_{1}^{-} {\to }^{{k}_{1}} I + {X}_{1}^{+} + {W}_{1}^{-} \qquad ( 1 c )$$

$${X}_{1}^{-} + {W}_{1}^{+} {\to }^{{k}_{1}} I + {X}_{1}^{-} + {W}_{1}^{+} \qquad ( 1 d )$$

$${X}_{2}^{+} + {W}_{2}^{+} {\to }^{{k}_{1}} {I}^{+} + {X}_{2}^{+} + {W}_{2}^{+} \qquad ( 2 a )$$

$${X}_{2}^{-} + {W}_{2}^{-} {\to }^{{k}_{1}} {I}^{+} + {X}_{2}^{-} + {W}_{2}^{-} \qquad ( 2 b )$$

$${X}_{2}^{+} + {W}_{2}^{-} {\to }^{{k}_{1}} {I}^{-} + {X}_{2}^{+} + {W}_{2}^{-} \qquad ( 2 c )$$

$${X}_{2}^{-} + {W}_{2}^{+} {\to }^{{k}_{1}} {I}^{-} + {X}_{2}^{-} + {W}_{2}^{+} \qquad ( 2 d )$$

$$⋮$$

$${X}_{N}^{+} + {W}_{N}^{+} {\to }^{{k}_{1}} {I}^{+} + {X}_{N}^{+} + {W}_{N}^{+} \qquad ( 3 a )$$

$${X}_{N}^{-} + {W}_{N}^{-} {\to }^{{k}_{1}} {I}^{+} + {X}_{N}^{-} + {W}_{N}^{-} \qquad ( 3 b )$$

$${X}_{N}^{+} + {W}_{N}^{-} {\to }^{{k}_{1}} {I}^{-} + {X}_{N}^{+} + {W}_{N}^{-} \qquad ( 3 c )$$

$${X}_{N}^{-} + {W}_{N}^{+} {\to }^{{k}_{1}} {I}^{-} + {X}_{N}^{-} + {W}_{N}^{+} \qquad ( 3 d )$$

$${Y}^{+} + {H}^{+} {\to }^{{k}_{1}} {I}^{-} + {H}^{+} \qquad ( 4 a )$$

$${Y}^{-} + {H}^{-} {\to }^{{k}_{1}} {I}^{+} + {H}^{-} \qquad ( 4 b )$$

$${I}^{+} + {C}^{+} {\to }^{{k}_{1}} {I}^{+} + {C}^{+} + {Y}^{+} \qquad ( 4 c )$$

$${I}^{-} + {C}^{-} {\to }^{{k}_{1}} {I}^{-} + {C}^{-} + {Y}^{+} \qquad ( 4 d )$$

$${X}_{1}^{+} + {X}_{1}^{-} {\to }^{{k}_{2}} Φ \qquad ( 5 a )$$

$${W}_{1}^{+} + {W}_{1}^{-} {\to }^{{k}_{2}} Φ \qquad ( 5 b )$$

$${X}_{2}^{+} + {X}_{2}^{-} {\to }^{{k}_{2}} Φ \qquad ( 5 c )$$

$${W}_{2}^{+} + {W}_{2}^{-} {\to }^{{k}_{2}} Φ \qquad ( 5 d )$$

$$⋮$$

$${X}_{i}^{+} + {X}_{i}^{-} {\to }^{{k}_{2}} Φ \qquad ( 6 a )$$

$${W}_{i}^{+} + {W}_{i}^{-} {\to }^{{k}_{2}} Φ \qquad ( 6 b )$$

$${Y}^{+} + {Y}^{-} {\to }^{{k}_{2}} Φ \qquad ( 6 c )$$

$${C}^{+} + {C}^{-} {\to }^{{k}_{2}} Φ \qquad ( 6 d )$$

$${H}^{+} + {H}^{-} {\to }^{{k}_{2}} Φ \qquad ( 6 e )$$

$${I}^{+} + {I}^{-} {\to }^{{k}_{2}} Φ \qquad ( 6 f )$$

Where Xᵢ(t)=[Xᵢ⁺]ₜ−[Xᵢ⁻]ₜ, i=1, 2, . . . , N, N represents the number of input items, [*]ₜ represents a concentration of a substance * at a time t, and Xᵢ represents an i-th input item, that is, X₁, X₂, . . . , X_{N} respectively represent the first input item, the second input item, . . . , the N-th input item of the machine learning (i.e., learning machine). Wᵢ(t)=[Wᵢ⁺]ₜ−[Wᵢ⁻]ₜ, Wᵢ represents an i-th weight, that is, W₁, W₂, . . . , W_{N} respectively represent the weight corresponding to the first input item, the weight corresponding to the second input item, . . . , the weight corresponding to the N-th input item. Xᵢ⁺ and Xᵢ⁻ respectively represent a positive part and a negative part of Xᵢ, specifically, X₁⁺, X₂⁺, . . . , X_{N}⁺ respectively represent positive parts of X₁, X₂, . . . , X_{N}; and X₁⁻, X₂⁻, . . . , X_{N}⁻ respectively represent negative parts of X₁, X₂, . . . , X_{N}. Apparently, [Xᵢ⁺]ₜ and [Xᵢ⁻]ₜ respectively represent concentrations of the positive part and the negative part of Xᵢ at the time t. Wᵢ⁺ and Wᵢ⁻ respectively represent a positive part and a negative part of Wᵢ, specifically, W₁⁺, W₂⁺, . . . , W_{N}⁺ respectively represent positive parts of W₁, W₂, . . . , W_{N}; and W₁, W₂, . . . , W_{N} respectively represent negative parts of W₁, W₂, . . . , W_{N}. Apparently, [Wᵢ⁺]ₜ and [Wᵢ⁻]ₜ respectively represent concentrations of the positive part and the negative part of Wᵢ at the time t. Y⁺ and Y⁻ respectively represent the positive part and the negative part of Y. H⁺ and H⁻ respectively represent the positive part and the negative part of H. C⁺ and C⁻ respectively represent the positive part and the negative part of C. I⁺ and I⁻ respectively represent the positive part and the negative part of I. k₁ and k₂ respectively represent different reaction rates. Φ represents a waste, which is meaningless to the machine learning. Supposing [H⁺]ₜ=[H⁻]ₜ≡1 nM, differential equations of concentrations varying over time of substances I⁺ and I⁻ are as follows:

$$\{ \begin{matrix}\frac{{d [ {I}^{+} ]}_{t}}{d t} = & {k}_{1} ( {{[ {W}_{1}^{+} ]}_{t} [ {X}_{1}^{+} ]}_{t} + {{[ {W}_{2}^{+} ]}_{t} [ {X}_{2}^{+} ]}_{t} + … + {{[ {W}_{N}^{+} ]}_{t} [ {X}_{N}^{+} ]}_{t} + \\ & {{[ {W}_{1}^{-} ]}_{t} [ {X}_{1}^{-} ]}_{t} + {{[ {W}_{2}^{-} ]}_{t} [ {X}_{2}^{-} ]}_{t} + … + {{[ {W}_{N}^{-} ]}_{t} [ {X}_{N}^{-} ]}_{t} + \\ & {{[ {Y}^{-} ]}_{t} [ {H}^{-} ]}_{t} ) \\ \frac{{d [ {I}^{-} ]}_{t}}{d t} = & {k}_{1} ( {{[ {W}_{1}^{+} ]}_{t} [ {X}_{1}^{-} ]}_{t} + {{[ {W}_{2}^{+} ]}_{t} [ {X}_{2}^{-} ]}_{t} + … + {{[ {W}_{N}^{+} ]}_{t} [ {X}_{N}^{-} ]}_{t} + \\ & {{[ {W}_{1}^{-} ]}_{t} [ {X}_{1}^{+} ]}_{t} + {{[ {W}_{2}^{-} ]}_{t} [ {X}_{2}^{+} ]}_{t} + … + {{[ {W}_{N}^{-} ]}_{t} [ {X}_{N}^{+} ]}_{t} + \\ & {{[ {Y}^{+} ]}_{t} [ {H}^{+} ]}_{t} )\end{matrix} \qquad ( 7 )$$

Where [Y⁺] and [Y⁻] respectively represent concentrations of the positive part and the negative part of Y at the time t; [I⁺]ₜ and [I⁻]^{t} respectively represent concentrations of the positive part and the negative part of I at the time t; [H⁺]ₜ and [H⁻]ₜ respectively represent concentrations of the positive part and the negative part of H at the time t.

The differential equations (7) are simplified as follows:

$$\{ \begin{matrix}\frac{{d [ {I}^{+} ]}_{t}}{d t} = {k}_{1} ( {\sum }_{i = 1}^{N} ( {{[ {W}_{i}^{+} ]}_{t} [ {X}_{i}^{+} ]}_{t} + {{[ {W}_{i}^{-} ]}_{t} [ {X}_{i}^{-} ]}_{t} ) + {{[ {Y}^{-} ]}_{t} [ {H}^{-} ]}_{t} ) \\ \frac{{d [ {I}^{-} ]}_{t}}{d t} = {k}_{1} ( {\sum }_{i = 1}^{N} ( {{[ {W}_{i}^{+} ]}_{t} [ {X}_{i}^{-} ]}_{t} + {{[ {W}_{i}^{-} ]}_{t} [ {X}_{i}^{+} ]}_{t} ) + {{[ {Y}^{+} ]}_{t} [ {H}^{+} ]}_{t} )\end{matrix}$$

$$and$$

$$\frac{{d [ I ]}_{t}}{d t} = \frac{{d [ {I}^{+} ]}_{t}}{d t} - \frac{{d [ {I}^{-} ]}_{t}}{d t} = {k}_{1} ( {\sum }_{i = 1}^{N} ( {{[ {W}_{i}^{+} ]}_{t} [ {X}_{i}^{+} ]}_{t} + {{[ {W}_{i}^{-} ]}_{t} [ {X}_{i}^{-} ]}_{t} - {{[ {W}_{i}^{+} ]}_{t} [ {X}_{i}^{-} ]}_{t} - {{[ {W}_{i}^{-} ]}_{t} [ {X}_{i}^{+} ]}_{t} + {{[ {Y}^{-} ]}_{t} [ {H}^{-} ]}_{t} - {{[ {Y}^{+} ]}_{t} [ {H}^{+} ]}_{t} ) )$$

Due to supposing [H⁺]ₜ=[H⁻]ₜ≡1 (nM) and

$$\frac{d I ( t )}{d t} = {k}_{1} ( {\sum }_{i = 1}^{N} {W}_{i} ( t ) {X}_{i} ( t ) - Y ( t ) ) .$$

When DNA hybridization reaction network reach dynamic equilibrium, and it can be concluded that:

$$Y ( t ) = {\sum }_{i = 1}^{N} {W}_{i} ( t ) {X}_{i} ( t ) .$$

Apparently. Y(t)=[Y⁺]ₜ−[Y⁻]ₜ, representing an output value of the system.

Reaction expressions of the algorithm part are as follows:

$${D}^{+} + {X}_{1}^{+} {\to }^{{k}_{3}} {D}^{+} + {X}_{1}^{+} + {W}_{1}^{+} \qquad ( 8 a )$$

$${D}^{-} + {X}_{1}^{-} {\to }^{{k}_{3}} {D}^{-} + {X}_{1}^{-} + {W}_{1}^{+} \qquad ( 8 b )$$

$${Y}^{+} + {X}_{1}^{-} {\to }^{{k}_{3}} {Y}^{+} + {X}_{1}^{-} + {W}_{1}^{+} \qquad ( 8 c )$$

$${Y}^{-} + {X}_{1}^{+} {\to }^{{k}_{3}} {D}^{-} + {X}_{1}^{+} + {W}_{1}^{+} \qquad ( 8 d )$$

$${D}^{+} + {X}_{1}^{-} {\to }^{{k}_{3}} {D}^{+} + {X}_{1}^{-} + {W}_{1}^{-} \qquad ( 8 e )$$

$${D}^{-} + {X}_{1}^{+} {\to }^{{k}_{3}} {D}^{-} + {X}_{1}^{+} + {W}_{1}^{-} \qquad ( 8 f )$$

$${Y}^{+} + {X}_{1}^{+} {\to }^{{k}_{3}} {Y}^{+} + {X}_{1}^{+} + {W}_{1}^{-} \qquad ( 8 g )$$

$${Y}^{-} + {X}_{1}^{-} {\to }^{{k}_{3}} {D}^{-} + {X}_{1}^{-} + {W}_{1}^{-} \qquad ( 8 h )$$

$${D}^{+} + {X}_{2}^{+} {\to }^{{k}_{3}} {D}^{+} + {X}_{2}^{+} + {W}_{2}^{+} \qquad ( 9 a )$$

$${D}^{-} + {X}_{2}^{-} {\to }^{{k}_{3}} {D}^{-} + {X}_{2}^{-} + {W}_{2}^{+} \qquad ( 9 b )$$

$${Y}^{+} + {X}_{2}^{-} {\to }^{{k}_{3}} {Y}^{+} + {X}_{2}^{-} + {W}_{2}^{+} \qquad ( 9 c )$$

$${Y}^{-} + {X}_{2}^{+} {\to }^{{k}_{3}} {D}^{-} + {X}_{2}^{+} + {W}_{2}^{+} \qquad ( 9 d )$$

$${D}^{+} + {X}_{2}^{-} {\to }^{{k}_{3}} {D}^{+} + {X}_{2}^{-} + {W}_{2}^{-} \qquad ( 9 e )$$

$${D}^{-} + {X}_{2}^{+} {\to }^{{k}_{3}} {D}^{-} + {X}_{2}^{+} + {W}_{2}^{-} \qquad ( 9 f )$$

$${Y}^{+} + {X}_{2}^{+} {\to }^{{k}_{3}} {Y}^{+} + {X}_{2}^{+} + {W}_{2}^{-} \qquad ( 9 g )$$

$${Y}^{-} + {X}_{2}^{-} {\to }^{{k}_{3}} {D}^{-} + {X}_{2}^{-} + {W}_{2}^{-} \qquad ( 9 h )$$

$$⋮$$

$${D}^{+} + {X}_{N}^{+} {\to }^{{k}_{3}} {D}^{+} + {X}_{N}^{+} + {W}_{N}^{+} \qquad ( 10 a )$$

$${D}^{-} + {X}_{N}^{-} {\to }^{{k}_{3}} {D}^{-} + {X}_{N}^{-} + {W}_{N}^{+} \qquad ( 10 b )$$

$${Y}^{+} + {X}_{N}^{-} {\to }^{{k}_{3}} {Y}^{+} + {X}_{N}^{-} + {W}_{N}^{+} \qquad ( 10 c )$$

$${Y}^{-} + {X}_{N}^{+} {\to }^{{k}_{3}} {D}^{-} + {X}_{N}^{+} + {W}_{N}^{+} \qquad ( 10 d )$$

$${D}^{+} + {X}_{N}^{-} {\to }^{{k}_{3}} {D}^{+} + {X}_{N}^{-} + {W}_{N}^{-} \qquad ( 10 e )$$

$${D}^{-} + {X}_{N}^{+} {\to }^{{k}_{3}} {D}^{-} + {X}_{N}^{+} + {W}_{N}^{-} \qquad ( 10 f )$$

$${Y}^{+} + {X}_{N}^{+} {\to }^{{k}_{3}} {Y}^{+} + {X}_{N}^{+} + {W}_{N}^{-} \qquad ( 10 g )$$

$${Y}^{-} + {X}_{N}^{+} {\to }^{{k}_{3}} {D}^{-} + {X}_{N}^{-} + {W}_{N}^{-} \qquad ( 10 h )$$

Where D⁺ and D⁻ respectively represent a positive part and a negative part of D; and k₃ represent a reaction rate which is different from k₁ and k₂.

Differential equations of concentrations varying over time of substances Wᵢ⁺ and Wᵢ⁻ are:

$$\{ \begin{matrix}\frac{d [ {W}_{1}^{+} ]}{d t} = {k}_{3} ( {{[ {D}^{+} ]}_{t} [ {X}_{1}^{+} ]}_{t} + {{[ {D}^{-} ]}_{t} [ {X}_{1}^{-} ]}_{t} + {{[ {Y}^{+} ]}_{t} [ {X}_{1}^{-} ]}_{t} + {{[ {Y}^{-} ]}_{t} [ {X}_{1}^{+} ]}_{t} ) \\ \frac{d [ {W}_{1}^{-} ]}{d t} = {k}_{3} ( {{[ {D}^{+} ]}_{t} [ {X}_{1}^{-} ]}_{t} + {{[ {D}^{-} ]}_{t} [ {X}_{1}^{+} ]}_{t} + {{[ {Y}^{+} ]}_{t} [ {X}_{1}^{+} ]}_{t} + {{[ {Y}^{-} ]}_{t} [ {X}_{1}^{-} ]}_{t} )\end{matrix} \qquad ( 11 )$$

$$\{ \begin{matrix}\frac{d [ {W}_{2}^{+} ]}{d t} = {k}_{3} ( {{[ {D}^{+} ]}_{t} [ {X}_{2}^{+} ]}_{t} + {{[ {D}^{-} ]}_{t} [ {X}_{2}^{-} ]}_{t} + {{[ {Y}^{+} ]}_{t} [ {X}_{2}^{-} ]}_{t} + {{[ {Y}^{-} ]}_{t} [ {X}_{2}^{+} ]}_{t} ) \\ \frac{d [ {W}_{2}^{-} ]}{d t} = {k}_{3} ( {{[ {D}^{+} ]}_{t} [ {X}_{2}^{-} ]}_{t} + {{[ {D}^{-} ]}_{t} [ {X}_{2}^{+} ]}_{t} + {{[ {Y}^{+} ]}_{t} [ {X}_{2}^{+} ]}_{t} + {{[ {Y}^{-} ]}_{t} [ {X}_{2}^{-} ]}_{t} )\end{matrix} \qquad ( 12 )$$

$$⋮$$

$$\{ \begin{matrix}\frac{d [ {W}_{N}^{+} ]}{d t} = {k}_{3} ( {{[ {D}^{+} ]}_{t} [ {X}_{N}^{+} ]}_{t} + {{[ {D}^{-} ]}_{t} [ {X}_{N}^{-} ]}_{t} + {{[ {Y}^{+} ]}_{t} [ {X}_{N}^{-} ]}_{t} + {{[ {Y}^{-} ]}_{t} [ {X}_{N}^{+} ]}_{t} ) \\ \frac{d [ {W}_{N}^{-} ]}{d t} = {k}_{3} ( {{[ {D}^{+} ]}_{t} [ {X}_{N}^{-} ]}_{t} + {{[ {D}^{-} ]}_{t} [ {X}_{N}^{+} ]}_{t} + {{[ {Y}^{+} ]}_{t} [ {X}_{N}^{+} ]}_{t} + {{[ {Y}^{-} ]}_{t} [ {X}_{N}^{-} ]}_{t} )\end{matrix} \qquad ( 13 )$$

Where D(t)=[D⁺]ₜ−[D⁻]ₜ represents an expectation value; and [D⁺]ₜ and [D⁻]ₜ respectively represent concentrations of the positive part and the negative part of D at the time t.

Concluding from the differential equations (11) to (13), what is obtained is:

$$\begin{matrix}\frac{d {W}_{1} ( t )}{dt} = \frac{{d [ {W}_{1}^{+} ]}_{t}}{dt} - \frac{{d [ {W}_{1}^{-} ]}_{t}}{dt} = {k}_{3} ( {{[ {D}^{+} ]}_{t} [ {X}_{1}^{+} ]}_{t} + {{[ {D}^{-} ]}_{t} [ {X}_{1}^{-} ]}_{t} - \\ {{[ {D}^{+} ]}_{t} [ {X}_{1}^{-} ]}_{t} - {{[ {D}^{-} ]}_{t} [ {X}_{1}^{+} ]}_{t} ) + \\ {k}_{2} ( {{[ {Y}^{+} ]}_{t} [ {X}_{1}^{-} ]}_{t} + {{[ {Y}^{-} ]}_{t} [ {X}_{1}^{+} ]}_{t} - \\ {{[ {Y}^{+} ]}_{t} [ {X}_{1}^{+} ]}_{t} + {{[ {Y}^{-} ]}_{t} [ {X}_{1}^{-} ]}_{t} ) \\ = {k}_{3} [ ( {[ {D}^{+} ]}_{t} - {[ {D}^{-} ]}_{t} ) - ( {[ {Y}^{+} ]}_{t} - {[ {Y}^{-} ]}_{t} ) ] \times \\ ( {[ {X}_{1}^{+} ]}_{t} - {[ {X}_{1}^{-} ]}_{t} ) \\ = {{k}_{3} ( {[ D ]}_{t} - {[ Y ]}_{t} ) [ {X}_{1} ]}_{t}\end{matrix}$$

$$\begin{matrix}\frac{d {W}_{2} ( t )}{dt} = \frac{{d [ {W}_{2}^{+} ]}_{t}}{dt} - \frac{{d [ {W}_{2}^{-} ]}_{t}}{dt} = {k}_{3} ( {{[ {D}^{+} ]}_{t} [ {X}_{2}^{+} ]}_{t} + {{[ {D}^{-} ]}_{t} [ {X}_{2}^{-} ]}_{t} - \\ {{[ {D}^{+} ]}_{t} [ {X}_{2}^{-} ]}_{t} - {{[ {D}^{-} ]}_{t} [ {X}_{2}^{*} ]}_{t} ) + \\ {k}_{2} ( {{[ {Y}^{+} ]}_{t} [ {X}_{2}^{-} ]}_{t} + {{[ {Y}^{-} ]}_{t} [ {X}_{2}^{+} ]}_{t} - \\ {{[ {Y}^{+} ]}_{t} [ {X}_{2}^{+} ]}_{t} + {{[ {Y}^{-} ]}_{t} [ {X}_{2}^{-} ]}_{t} ) \\ = {k}_{3} [ ( {[ {D}^{+} ]}_{t} - {[ {D}^{-} ]}_{t} ) - ( {[ {Y}^{+} ]}_{t} - {[ {Y}^{-} ]}_{t} ) ] \times \\ ( {[ {X}_{2}^{+} ]}_{t} - {[ {X}_{2}^{-} ]}_{t} ) \\ = {{k}_{3} ( {[ D ]}_{t} - {[ Y ]}_{t} ) [ {X}_{2} ]}_{t}\end{matrix}$$

$$⋮$$

$$\begin{matrix}\frac{{dW}_{N} ( t )}{dt} = \frac{{d [ {W}_{N}^{+} ]}_{t}}{dt} - \frac{{d [ {W}_{N}^{-} ]}_{t}}{dt} = {k}_{3} ( {{[ {D}^{+} ]}_{t} [ {X}_{N}^{+} ]}_{t} + {{[ {D}^{-} ]}_{t} [ {X}_{N}^{-} ]}_{t} - \\ {{[ {D}^{+} ]}_{t} [ {X}_{N}^{-} ]}_{t} - {{[ {D}^{-} ]}_{t} [ {X}_{N}^{*} ]}_{t} ) + \\ {k}_{2} ( {{[ {Y}^{+} ]}_{t} [ {X}_{N}^{-} ]}_{t} + {{[ {Y}^{-} ]}_{t} [ {X}_{N}^{*} ]}_{t} \\ = {k}_{3} [ ( {[ {D}^{+} ]}_{t} - {[ {D}^{-} ]}_{t} ) - ( {[ {Y}^{+} ]}_{t} - {[ {Y}^{-} ]}_{t} ) ] \times \\ ( {[ {X}_{N}^{+} ]}_{t} - {[ {X}_{N}^{-} ]}_{t} ) \\ = {{k}_{3} ( {[ D ]}_{t} - {[ Y ]}_{t} ) [ {X}_{N} ]}_{t}\end{matrix} .$$

Reaction expressions of the testing part of machine learning are as follows:

$${X}_{1}^{+} + {\hat{W}}_{1}^{+} {⟶}^{{k}_{1}} {\hat{I}}^{+} + {X}_{1}^{+} + {\hat{W}}_{1}^{+} \qquad ( 14 a )$$

$${X}_{1}^{-} + {\hat{W}}_{1}^{-} {⟶}^{{k}_{1}} {\hat{I}}^{+} + {X}_{1}^{-} + {\hat{W}}_{1}^{+} \qquad ( 14 b )$$

$${X}_{1}^{+} + {\hat{W}}_{1}^{-} {⟶}^{{k}_{1}} {\hat{I}}^{-} + {X}_{1}^{+} + {\hat{W}}_{1}^{-} \qquad ( 14 c )$$

$${X}_{1}^{-} + {\hat{W}}_{1}^{+} {⟶}^{{k}_{1}} {\hat{I}}^{-} + {X}_{1}^{-} + {\hat{W}}_{1}^{+} \qquad ( 14 d )$$

$${X}_{2}^{+} + {\hat{W}}_{2}^{+} {⟶}^{{k}_{1}} {\hat{I}}^{+} + {X}_{2}^{+} + {\hat{W}}_{2}^{+} \qquad ( 15 a )$$

$${X}_{2}^{+} + {\hat{W}}_{2}^{+} {⟶}^{{k}_{1}} {\hat{I}}^{+} + {X}_{2}^{+} + {\hat{W}}_{2}^{+} \qquad ( 15 b )$$

$${X}_{2}^{+} + {\hat{W}}_{2}^{-} {⟶}^{{k}_{1}} {\hat{I}}^{-} + {X}_{2}^{+} + {\hat{W}}_{2}^{-} \qquad ( 15 c )$$

$${X}_{2}^{-} + {\hat{W}}_{2}^{+} {⟶}^{{k}_{1}} {\hat{I}}^{-} + {X}_{2}^{-} + {\hat{W}}_{2}^{+} \qquad ( 15 d )$$

$$⋮$$

$${X}_{N}^{+} + {\hat{W}}_{N}^{+} {⟶}^{{k}_{1}} {\hat{I}}^{+} + {X}_{N}^{+} + {\hat{W}}_{N}^{+} \qquad ( 16 a )$$

$${X}_{N}^{-} + {\hat{W}}_{N}^{-} {⟶}^{{k}_{1}} {\hat{I}}^{+} + {X}_{N}^{-} + {\hat{W}}_{N}^{-} \qquad ( 16 b )$$

$${X}_{N}^{+} + {\hat{W}}_{N}^{-} {⟶}^{{k}_{1}} {\hat{I}}^{-} + {X}_{N}^{+} + {\hat{W}}_{N}^{-} \qquad ( 16 c )$$

$${X}_{N}^{-} + {\hat{W}}_{N}^{+} {⟶}^{{k}_{1}} {\hat{I}}^{-} + {X}_{N}^{-} + {\hat{W}}_{N}^{+} \qquad ( 16 d )$$

$${\hat{Y}}^{+} + {\hat{H}}^{+} {⟶}^{{k}_{1}} {\hat{I}}^{-} + {\hat{H}}^{+} \qquad ( 17 a )$$

$${\hat{Y}}^{-} + {\hat{H}}^{-} {⟶}^{{k}_{1}} {\hat{I}}^{+} + {\hat{H}}^{-} \qquad ( 17 b )$$

$${\hat{I}}^{+} + {\hat{C}}^{+} {⟶}^{{k}_{1}} {\hat{I}}^{+} + {\hat{C}}^{+} + {\hat{Y}}^{+} \qquad ( 17 c )$$

$${\hat{I}}^{-} + {\hat{C}}^{-} {⟶}^{{k}_{1}} {\hat{I}}^{-} + {\hat{C}}^{-} + {\hat{Y}}^{+} \qquad ( 17 d )$$

$${X}_{i}^{+} + {X}_{i}^{-} {⟶}^{{k}_{2}} Φ \qquad ( 18 a )$$

$${\hat{W}}_{i}^{+} + {\hat{W}}_{i}^{-} {⟶}^{{k}_{2}} Φ \qquad ( 18 b )$$

$${\hat{Y}}^{+} + {\hat{Y}}^{-} {⟶}^{{k}_{2}} Φ \qquad ( 18 c )$$

$${\hat{C}}^{+} + {\hat{C}}^{-} {⟶}^{{k}_{2}} Φ \qquad ( 18 d )$$

$${\hat{H}}^{+} + {\hat{H}}^{-} {⟶}^{{k}_{2}} Φ \qquad ( 18 e )$$

$${\hat{I}}^{+} + {\hat{I}}^{-} {⟶}^{{k}_{2}} Φ \qquad ( 18 f )$$

Where Ŵᵢ(t)=[Ŵᵢ⁺]ₜ−[Ŵᵢ⁻]ₜ Ŵᵢ represents a weight obtained after the rounds of training, that is, Ŵ₁, Ŵ₂, . . . , Ŵ_{N} represent weights obtained after the rounds of learning, which are also weights used in the testing part. Ŵᵢ⁺ and Ŵᵢ⁻ respectively represent the positive part and the negative part of Ŵᵢ. Ŷ represents an output of the testing part; Ŷ⁺ and Ŷ⁻ respectively represent the positive part and the negative part of Ŷ; Î, Ĥ and Ĉ represent substances involved in chemical reaction in the testing part; Ĥ⁺ and Ĥ⁻ respectively represent the positive part and the negative part of Ĥ; Ĉ⁺ and Ĉ⁻ respectively represent the positive part and the negative part of Ĉ; Î⁺ and Î⁻ respectively represent the positive part and the negative part of Î.

Concluded from the reaction equations (14a) to (18f), differential equations of concentrations varying over time of substances Î⁺ and Î⁻ are:

$$\{ \begin{matrix}\frac{{d [ {\hat{I}}^{+} ]}_{t}}{dt} = {k}_{1} ( {\sum }_{i = 1}^{N} ( {{[ {\hat{W}}_{i}^{+} ]}_{t} [ {X}_{i}^{+} ]}_{t} + {{[ {\hat{W}}_{i}^{-} ]}_{t} [ {X}_{i}^{-} ]}_{t} ) + {{[ {\hat{Y}}^{-} ]}_{t} [ {\hat{H}}^{-} ]}_{t} ) \\ \frac{{d [ {\hat{I}}^{-} ]}_{t}}{dt} = {k}_{1} ( {\sum }_{i = 1}^{N} ( {{[ {\hat{W}}_{i}^{+} ]}_{t} [ {X}_{i}^{-} ]}_{t} + {{[ {\hat{W}}_{i}^{-} ]}_{t} [ {X}_{i}^{+} ]}_{t} ) + {{[ {\hat{Y}}^{+} ]}_{t} [ {\hat{H}}^{+} ]}_{t} )\end{matrix}$$

What can be concluded from above differential equations is:

$$\frac{d \hat{I}}{dt} = \frac{{d [ {\hat{I}}^{+} ]}_{t}}{dt} - \frac{{d [ {\hat{I}}^{-} ]}_{t}}{dt} = {k}_{1} ( {\sum }_{i = 1}^{N} ( {{[ {\hat{W}}_{i}^{+} ]}_{t} [ {X}_{i}^{+} ]}_{t} + {{[ {\hat{W}}_{i}^{-} ]}_{t} [ {X}_{i}^{-} ]}_{t} - {{[ {\hat{W}}_{i}^{+} ]}_{t} [ {X}_{i}^{-} ]}_{t} + {{[ {\hat{W}}_{i}^{-} ]}_{t} [ {X}_{i}^{+} ]}_{t} ) - ( {{[ {\hat{γ}}^{+} ]}_{t} [ {\hat{H}}^{+} ]}_{t} - {{[ \hat{γ} ]}_{t} [ {\hat{H}}^{-} ]}_{t} ) )$$

When [Ĥ⁺]ₜ=[Ĥ⁻]^{t}≡1 nM, what can be obtained is:

$$\frac{d \hat{I}}{dt} = {k}_{1} ( {\sum }_{i = 1}^{N} {\hat{W}}_{i} {X}_{i} - \hat{Y} ) .$$

When Î⁺ and Î⁻ reach dynamic equilibrium,

$$\frac{df}{dt} = 0 ,$$

then:

$$\hat{Y} = {\sum }_{i = 1}^{N} {\tilde{W}}_{i} {X}_{i} .$$

Where Ŷ(t)=[Ŷ⁺]ₜ−[Ŷ⁻]ₜ, Ŷ represents the output of machine learning.

It should be noted that the catalytic reaction module 1, the catalytic reaction module 2, and the annihilation reaction module in the training part, the algorithm part, and the testing part belong to a same type of reaction module, but do not belong to a same reaction module. For example, from differential equations of concentrations varying over time of î⁺ and Î⁻, it can be seen that the symbol has a subscript i below it. When i changes, the DNA molecules undergo changes accordingly, which means that all the reaction modules provided in the present disclosure do not refer to any particular one reaction, but describe one type of reaction.

The catalytic reaction module 1 (catalytic reaction module 2) in the training and testing parts belong to the same type of the catalytic reaction module 1 (catalytic reaction module 2). The identification symbols of the catalytic reaction module 1 (catalytic reaction module 2) in the training and testing parts are different, suggesting different DNA molecules are used, and the identification symbols in the testing part have pointed caps (i.e., \hat{ }), while those in the training part does not.

### 2. Implementing Linear Machine Learning by Using DNA Molecular Circuits.

Due to the fact that reactants and products in idealized reactions are abstract substances rather than specific biochemical substances, while DNA hybridization reactions can achieve any type of idealized reaction. The DNA hybridization reactions are utilized to implement the linear learning machine.

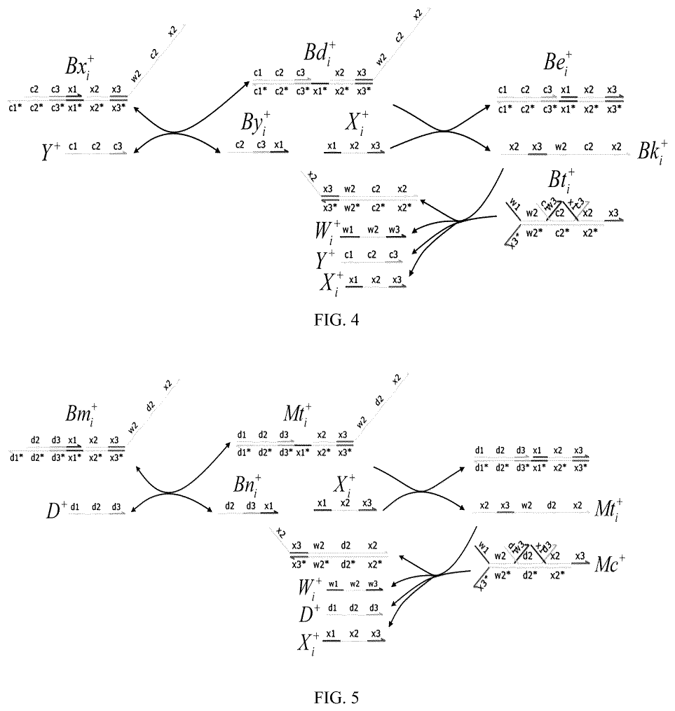

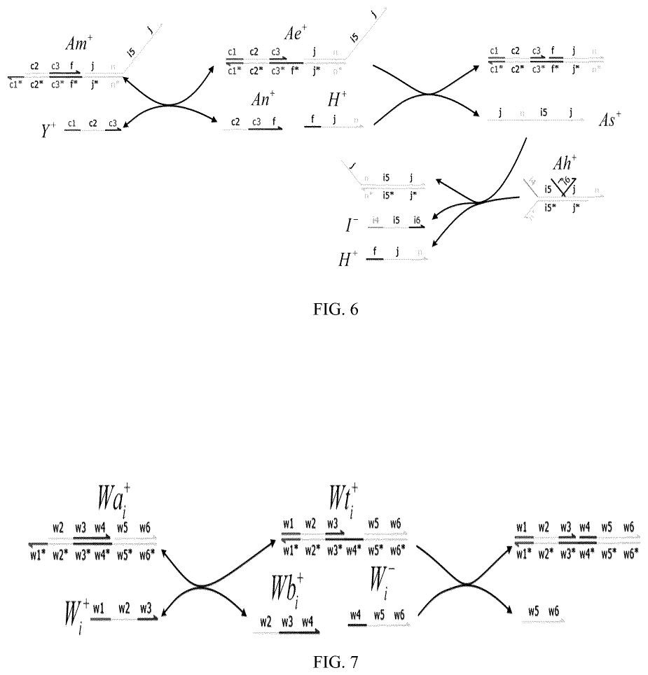

The reaction equations (1) to (18) can be classified into several types of reactions: as shown in FIG. 2, the reaction equations (1a) to (3d) and (13a) to (15d) belong to the submodule 1 of the catalytic reaction module 1. As illustrated in FIG. 3, the reaction equations (4c), (4d), (17c), and (17d) belong to a first type of catalytic reaction, which can be achieved by the submodule 2 of the catalytic reaction module 1. As illustrated in FIG. 4, the reaction equations (8c), (8d), (8g), (8h), (9c), (9d), (9g), (9h), (10c), (10d), (10g), and (10h) belong to the first type of catalytic reaction, which can be achieved by the submodule 3 of the catalytic reaction module 1. As illustrated in FIG. 5, the reaction equations (8a), (8b), (8e), (8f), (9a), (9b), (9e), (9f), (10a), (10b), (10e), and (10f) belong to the first type of catalytic reaction, which can be achieved by the submodule 4 of the catalytic reaction module 1. As illustrated in FIG. 6, the reaction equations (4a), (4b), (17a) and (17b) belong to a second type of catalytic reaction. The reaction equations (5a) to (6f) and (18a) to (18f) belong to the annihilation reaction type and can be achieved through the annihilation reaction module. Due to the homogeneity and cascading nature of these reaction modules, they can be cascaded into DNA molecular circuits to achieve the machine learning system. The three types of DNA reaction modules are described as follows:

### (1) The Catalytic Reaction Module 1

The idealized reaction equation for the catalytic reaction module 1 is

$${X}_{i}^{+} + {W}_{i}^{+} {⟶}^{{k}_{i}} {I}^{+} + {X}_{i}^{+} + {W}_{i}^{+} ,$$

which is obtained by following DNA strand displacement reactions:

$$\{ \begin{matrix}{W}_{i}^{+} + {Ap}_{i}^{+} {⇄}_{{q}_{m}}^{{q}_{i}} {Ad}_{i}^{+} + {Aq}_{i}^{+} \\ {Ad}_{i}^{+} + {X}_{i}^{+} {⟶}^{{q}_{m}} {Ac}_{i}^{+} + waste \\ {Ac}_{i}^{+} + {Ab}_{i}^{+} {⟶}^{{q}_{m}} {I}^{+} + {X}_{i}^{+} + {W}_{i}^{+} + waste\end{matrix} \qquad ( 19 )$$

Where I⁺ is catalyzed, Xᵢ represents an input signal DNA molecule, and Wᵢ⁺ represents a weight reporting strand. Apᵢ⁺, Aqᵢ⁺ and Abᵢ⁺ are auxiliary DNA strands and initial concentrations of the auxiliary DNA strands are Cₘ, meeting Cₘ≥[Xᵢ³⁰]₀, [Wᵢ⁺]₀, [I⁺]₀. A reaction rate qᵢ and kᵢ meet qᵢ≤qₘ, kᵢ=qᵢ, and qₘ represents a maximum reaction rate. The DNA implementation of the submodule 1 of the catalytic reaction module 1 is illustrated in FIG. 2. As illustrated in FIGS. 3, 4, 5, the DNA implementation process of the submodules 2-4 of the catalytic reaction module 1 is similar to the submodule 1.

### (2) The Catalytic Reaction Module 2

The idealized reaction equation of the catalytic reaction module 2 is:

$${Y}^{+} + {H}^{+} {⟶}^{{k}_{i}} {I}^{-} + {H}^{+} ,$$

which is obtained by following DNA strand displacement reactions:

$$\{ \begin{matrix}{Y}^{+} + {Am}^{+} {⇄}_{{q}_{m}}^{{q}_{i}} {An}^{+} + {Ae}^{+} \\ {Ae}^{+} + {H}^{+} {⟶}^{{q}_{m}} {As}^{+} + waste \\ {As}^{+} + {Ah}^{+} {⟶}^{{q}_{m}} {H}^{+} + {I}^{-} + waste\end{matrix} \qquad ( 20 )$$

Where I⁻ is catalyzed, Am⁺, An⁺, Ah⁺ and H⁺ are auxiliary DNA strands and initial concentrations of the auxiliary DNA strands Am⁺, An⁺ and Ah⁺ are respectively set as [Am⁺]₀=[An⁺]₀=[Ah⁺]₀=Cₘ, meeting Cₘ□[Y⁺]₀,[I⁻]₀. An initial concentration of H⁺ is 1 nM. The reaction rate qᵢ meets qᵢ≤qₘ, kᵢ=qᵢ. The DNA implementation of the catalytic reaction module 2 is illustrated in FIG. 6.

### (3) The Annihilation Reaction Module

The idealized reaction equation of the annihilation reaction module is:

$${W}_{i}^{+} + {W}_{i}^{-} {⟶}^{{k}_{i}} Φ ,$$

which is obtained by following DNA strand displacement reactions:

$$\{ \begin{matrix}{W}_{i}^{+} + {Wa}_{i}^{+} {⇄}_{{q}_{m}}^{{q}_{i}} {Wb}_{i}^{+} + {Wt}_{i}^{+} \\ {Wt}_{i}^{+} + {W}_{i}^{-} {⟶}^{{q}_{m}} Φ\end{matrix} \qquad ( 21 )$$

Where Wᵢ⁺ and Wᵢ⁻ are annihilated, Waᵢ⁺ and Wbᵢ⁺ are auxiliary DNA strands and initial concentrations of the auxiliary DNA strands are Cₘ, meeting Cₘ□[Wᵢ⁺]₀, [Wᵢ⁻]₀. The reaction rate qᵢ meets qᵢ≤qₘ, kᵢ=qᵢ. The DNA implementation of the annihilation reaction module is illustrated in FIG. 7.

3. Training of linear machine learning.

This section belongs to the application of the machine learning system. The molecular learning machine has the ability to predict the relationship between the total current and voltage of parallel circuits, which is obtained through data training. Therefore, a third part is the training of the machine learning system. In order to test the learning ability of the molecular learning machine, it is necessary to test the molecular learning machine.

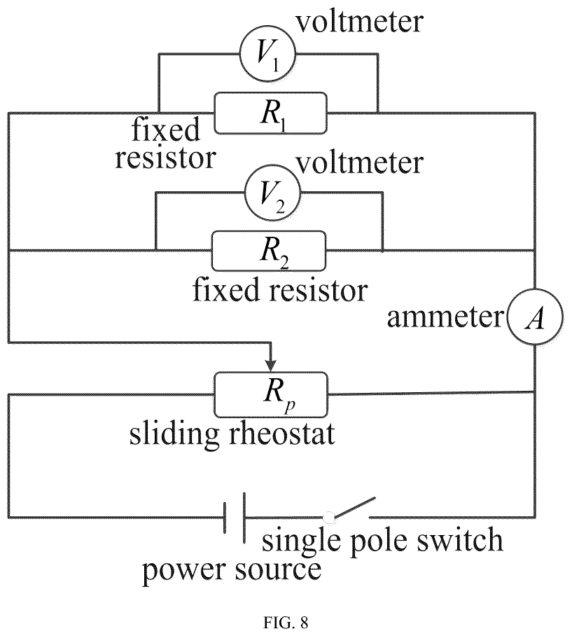

As illustrated in FIG. 8, the resistance value of the sliding rheostat is adjusted to measure the voltages U₁, U₂ and the total current I. The two voltage values and one total current are used as a set of training data. By adjusting the sliding rheostat, another set of training data can be obtained. These obtained training data are inputted into the DNA molecular machine learning system. Through the processing of the DNA molecular learning machine algorithm, weights are updated and the relative error between the output of the DNA molecular learning machine and the expected value is calculated. When the relative error reaches or is lower than the set value of 0.2, the training goal is achieved and the training is stopped. The weights obtained through training are the value of w₁, w₂, . . . w_{N} of the linear function relationship I(U₁, U₂, . . . . U_{N})=w₁U₁+w₂U₂+ . . . +w_{N}U_{N}. The functional relationship between them can be fitted by using the voltage and current values when the value of w₁, w₂, . . . w_{N} are obtained. These parameter values correspond to the reciprocals of the fixed resistance (the relationship between partial voltage and total current in a parallel circuit is

$$I = \frac{{U}_{1}}{{R}_{1}} + \frac{{U}_{2}}{{R}_{2}} + … + \frac{{U}_{3}}{{R}_{3}} .$$

Therefore, the DNA molecule learning machine can predict the partial resistance value R₁, R₂, . . . R_{N}.

The present disclosure utilizes a DNA linear learning machine to learn the relationship between the total current and voltages of a parallel circuit I(U₁, U₂, . . . U_{N})=w₁U₁+w₂U₂+ . . . +w_{N}U_{N}. The weights wᵢ and inputs Uᵢ(i=1, 2, . . . N) are both real numbers. As the weights and inputs are represented by the concentration of DNA strands, wᵢ, Uᵢ, I≥0.

### (1) Training of Linear Machine Learning.

The training of machine learning includes multiple rounds of training. One round of training includes M groups of training data, each group of which is composed of N data, that is, the machine learning has N inputs and i=1, 2, . . . , N. The training data set is shuffled to obtain another round of training data. In the first round of training data, Ψᵢ=[αᵢ(1,1), αᵢ(2,1), . . . , αᵢ(M, 1)] and Γ={\tilde{d}₁(1), \tilde{d}₁(2), . . . , \tilde{d}₁ (M)} respectively represent partial voltage and total current. The data can be normalized as follows:

$$\{ \begin{matrix}{\hat{x}}_{i} ( k , l ) = ρ {α}_{i} ( k , l ) / η \\ {\hat{d}}_{l} ( k ) = ρ {\tilde{d}}_{l} ( k ) / η\end{matrix} \qquad ( 22 )$$

$$where$$

$$\{ \begin{matrix}{η}_{i} = max ( {Ψ}_{i} ) - min ( {Ψ}_{i} ) \\ η = max ( [ {η}_{1} , {η}_{2} , … {η}_{N} ] ) \\ {\tilde{d}}_{l} ( k ) = {\sum }_{i = 1}^{N} {w}_{i} {x}_{i} ( l , k )\end{matrix} .$$

αᵢ(k,l) represents the i-th data of the k-th group of data in the l-th round of training, k=1, 2, . . . , M, l=1, 2, . . . , Λ and ρ are positive adjustment parameters. Ψᵢ=[xᵢ(1), xᵢ(2), . . . , xᵢ(K)]. \hat{x}ᵢ(k,l) and \hat{d}ₗ (k) are the initial concentration setting of the input signal DNA strand for machine learning. The first round of training data meets \hat{x}1 (k,l)=[Xᵢₖ⁺]₀−[Xᵢₖ^{-]}₀ and d₁(k)=[Dₖ⁺]₀−[Dₖ⁻]₀, shuffling the order can obtain training data for other rounds.

### (2) Assessment of Training of Linear Machine Learning.

In the l-th training, a relative error eₗ(k) is defined as follows.

$${e}_{l} ( k ) = \frac{{D}_{l} ( k ) - {\hat{d}}_{l} ( k )}{{\hat{d}}_{l} ( k )} \qquad ( 23 )$$

$$where$$

$$\{ \begin{matrix}{D}_{l} ( k ) = ψ ( {Y}_{1} ( k , l ) ) \\ {Y}_{1} ( k , l ) = {{\sum }_{n = 1}}^{L} {\hat{V}}_{n 1} ( l ) {y}_{n} ( k , l ) \\ {y}_{n} ( k , l ) = ψ ( {S}_{n} ( k , l ) ) \\ {S}_{n} ( k , l ) = {{\sum }_{i = 1}}^{N} {\hat{W}}_{in} ( l ) {x}_{i} ( k , l )\end{matrix}$$

Ŵᵢₙ (l) and \hat{V}ₙ₁ (l) represent the weights of the input layer and hidden layer obtained after the l-th training.

To assess the training results, the average relative error is defined as follows:

$${\overline{e}}_{l} = \frac{1}{K} {{\sum }_{k = 1}}^{K} \vert {e}_{l} ( k ) \vert \qquad ( 24 )$$

After multiple trainings, when the average relative error reaches the target value, the training will be stopped.

As illustrated in FIG. 8, a nonlinear neural network with two input nodes is taken as an example to illustrate the training and assessment of the relationship between voltage and total current in a parallel circuit containing two fixed resistors based on DNA strand displacement reaction neural network.

Raw training data is U₁∈{1.2V, 1.4V, 1.6V, . . . , 5.0V}, U₂∈{1.3V, 1.5V, 1.7V, . . . , 5.1V} and I∈{0.37A, 0.43A, 0.49A, . . . , 1.51A} and there are 20 sets of data in total. The value of ρ is that ρ=3. Initial concentrations of DNA strands X₁⁺, X₁⁻, X₂⁺ and X₂⁻ are set as [X₁⁺]₀∈{2.2632 nM, 2.4211 nM, 2.5789 nM, . . . , 5.2632 nM} [X₁⁻]₀∈{1.6316 nM, 1.6842 nM, 1.7368 nM, . . . , 2.6316 nM}, [X₂⁺]₀∈{2.3158 nM, 2.4737 nM, 2.6316 nM, . . . , 5.3158 nM}, [X₂⁻]₀∈{1.6316 nM, 1.6842 nM, 1.7368 nM, . . . , 2.6316 nM} Initial concentration of auxiliary DNA molecules and reaction rates are set in Table 1.

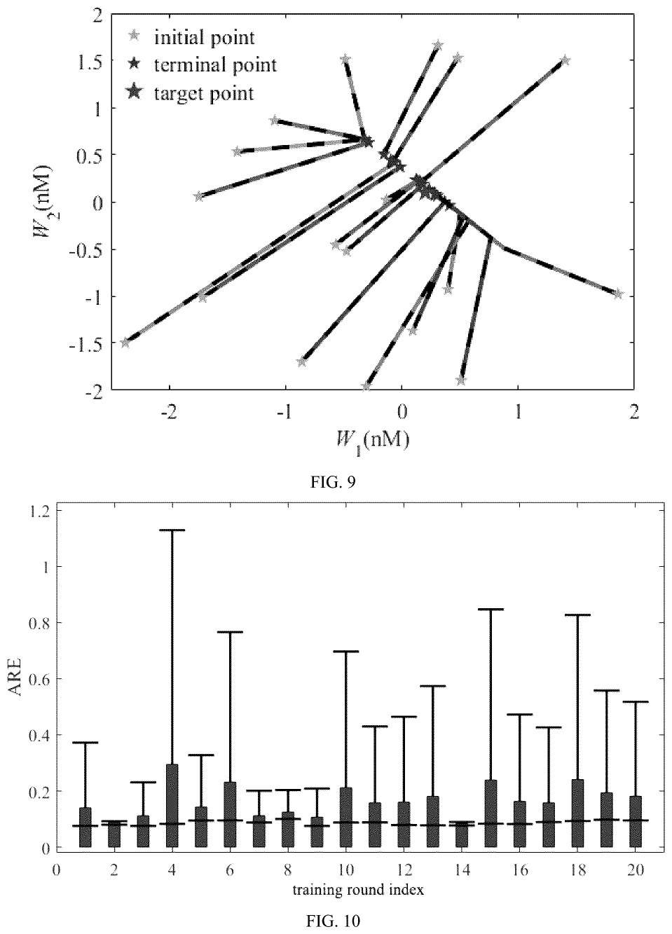

FIG. 9 illustrated the update trajectory of weights during 20 rounds of training. It is evident that after training, the endpoint value of weights is very close to the target value, indicating that the linear learning machine has good learning ability.

As illustrated in FIG. 10, in 20 rounds of training, the average relative error is all around 0.2, and the training goal is basically achieved.

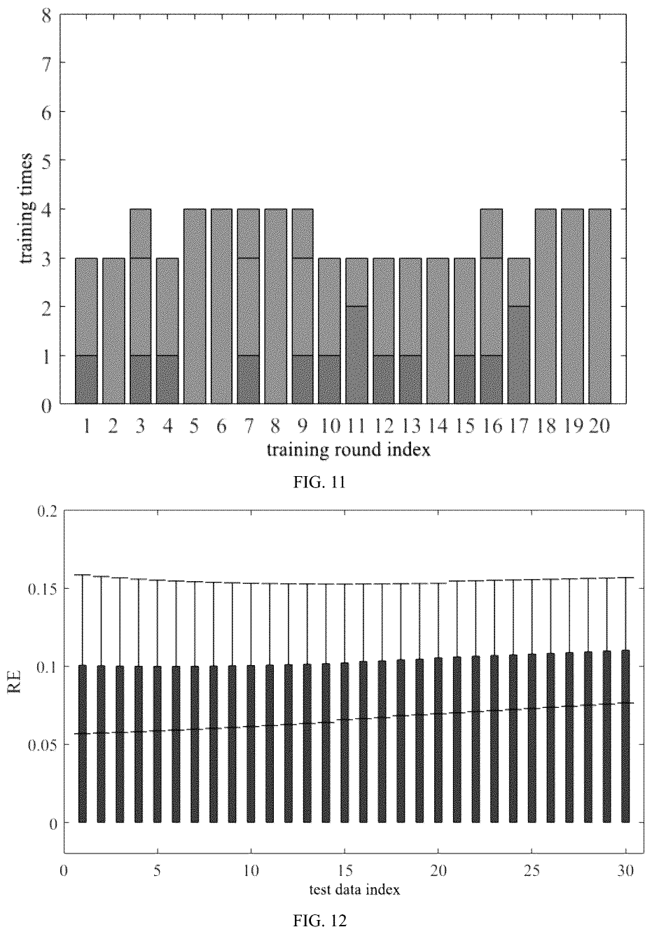

FIG. 11 illustrated a total number of training times required to achieve the training objectives in 20 rounds of training, which is clearly 4.

TABLE 1
concentrations of DNA strands and reaction rate settings

| Reaction rate | Value | Concentration | Value | |
|---|---|---|---|---|
| q₁ | 0.01/nM/s | [I⁺]₀ | 2 | nM |
| q₂ | 0.001/nM/s | [I⁻]₀ | 2 | nM |
| q₃ | 0.001/nM/s | [Y⁺]₀ | 20 | nM |
| qₘ | 1.0/nM/s | [Y⁻]₀ | 20 | nM |
| | | [C⁺]₀ | 20 | nM |
| | | [C⁻]₀ | 20 | nM |
| | | [H⁺]₀ | 1 | nM |
| | | [H⁻]₀ | 1 | nM |
| | | [Î⁺]₀ | 2 | nM |
| | | [Î⁻]₀ | 2 | nM |
| | | [Ŷ⁺]₀ | 100 | nM |
| | | [Ŷ⁻]₀ | 100 | nM |
| | | [Ĉ⁺]₀ | 200 | nM |
| | | [Ĉ⁻]₀ | 200 | nM |
| | | Cₘ | 2000 | nM |

### (3) Testing and Assessment of Linear Machine Learning.

The testing of machine learning includes multiple rounds of testing. One round of testing includes P sets of training data. The test data sets are shuffled to obtain another round of test data. In the first round of testing, the partial resistance and total voltage values are represented by Φᵢ=[βᵢ(1,1), βᵢ(2,1), . . . , Bᵢ(P,1)] and Γ={\tilde{d}′₁(1), \tilde{d}′₁(2), . . . , \tilde{d}′₁(M)} respectively. The data can be normalized as follows.

$$\{ \begin{matrix}{\hat{x}}_{i}^{′} ( p , g ) = ρ {β}_{i} ( p , g ) / θ \\ {\hat{d}}_{g}^{′} ( p ) = ρ {\tilde{d}}_{g}^{′} ( p ) / θ\end{matrix} \qquad ( 25 )$$

Where βᵢ(p,g) represents an i-th data of a p-th set of data in a g-th round of training. p=1, 2, . . . , P, g=1, 2, . . . , G, θᵢ=max(Φᵢ)−min(Φᵢ), θ=max([θ₁, θ₂, . . . θ_{N}]) and \tilde{d}′₁(p)=Σᵢ₌₁^{N}wᵢxᵢ(g,p).

In the g-th round of testing, the relative error e′_{g}(p) is defined as follows.

$${e}_{g}^{′} ( p ) = \frac{{\tilde{y}}_{g}^{′} ( p ) - {\hat{d}}_{g}^{′} ( p )}{{\hat{d}}_{l}^{′} ( p )} \qquad ( 26 )$$

$$where$$

$${\hat{y}}_{g} ( p ) = {\sum }_{i = 1}^{N} {\hat{W}}_{i} {\hat{x}}_{i}^{′} ( k ) .$$

\tilde{w}ᵢ represents the weight obtained after this round of training.

To assess the results of this round of training, the average relative error during the testing phase is defined as follows:

$${\overline{e}}_{g}^{′} = \frac{1}{P} {\sum }_{p = 1}^{P} \vert {e}_{g} ( k ) \vert \qquad ( 27 )$$

Still taking the nonlinear neural network with two input nodes as an example to illustrate the test results of the neural network based on DNA strand replacement reaction. The raw data for the test are as follows.

U₁∈{1.27V, 1.31V, 1.34V, . . . , 1.90V}, U₂∈{1.24V, 1.28V, 1.31V, . . . , 1.86V} and I∈{0.77A, 0.80A, 0.83A, . . . , 1.64A} and there are 30 sets of data in total. As illustrated in FIG. 12, average relative error of 30 sets of data in each round of 20 rounds of testing are all about 0.1, indicating the neural network based on DNA strand displacement reaction basically meets the testing requirements.

The above embodiments are only used to illustrate the technical solution of the present disclosure, and not to limit it. Although the present disclosure has been described in detail with reference to the aforementioned embodiments, those skilled in the art should understand that they can still modify the technical solutions recorded in the aforementioned embodiments, or equivalently replace some or all of the technical features. And these modifications or replacements do not make the essence of the corresponding technical solutions deviate from the scope of the technical solutions of the various embodiments of the present disclosure.

What is claimed is:

1. A linear machine learning method based on deoxyribonucleic acid (DNA) hybridization reaction technology, comprising: a training part, an algorithm part and a testing part; wherein,
(1) reaction expressions of the training part are as follows:
$${X}_{1}^{+} + {W}_{1}^{+} {\to }^{{k}_{1}} {I}^{+} + {X}_{1}^{+} + {W}_{1}^{+} \qquad ( 1 a )$$
$${X}_{1}^{-} + {W}_{1}^{-} {\to }^{{k}_{1}} {I}^{+} + {X}_{1}^{-} + {W}_{1}^{-} \qquad ( 1 b )$$
$${X}_{1}^{+} + {W}_{1}^{-} {\to }^{{k}_{1}} {I}^{-} + {X}_{1}^{+} + {W}_{1}^{-} \qquad ( 1 c )$$
$${X}_{1}^{-} + {W}_{1}^{+} {\to }^{{k}_{1}} {I}^{-} + {X}_{1}^{-} + {W}_{1}^{+} \qquad ( 1 d )$$
$${X}_{2}^{+} + {W}_{2}^{+} {\to }^{{k}_{1}} {I}^{+} + {X}_{2}^{+} + {W}_{2}^{+} \qquad ( 2 a )$$
$${X}_{2}^{-} + {W}_{2}^{-} {\to }^{{k}_{1}} {I}^{+} + {X}_{2}^{-} + {W}_{2}^{-} \qquad ( 2 b )$$
$${X}_{2}^{+} + {W}_{2}^{-} {\to }^{{k}_{1}} {I}^{-} + {X}_{2}^{+} + {W}_{2}^{-} \qquad ( 2 c )$$
$${X}_{2}^{-} + {W}_{2}^{+} {\to }^{{k}_{1}} {I}^{-} + {X}_{2}^{-} + {W}_{2}^{+} \qquad ( 2 d )$$
$$⋮$$
$${X}_{N}^{+} + {W}_{N}^{+} {\to }^{{k}_{1}} {I}^{+} + {X}_{N}^{+} + {W}_{N}^{+} \qquad ( 3 a )$$
$${X}_{N}^{-} + {W}_{N}^{-} {\to }^{{k}_{1}} {I}^{+} + {X}_{N}^{-} + {W}_{N}^{-} \qquad ( 3 b )$$
$${X}_{N}^{+} + {W}_{N}^{-} {\to }^{{k}_{1}} {I}^{-} + {X}_{N}^{+} + {W}_{N}^{-} \qquad ( 3 c )$$
$${X}_{N}^{-} + {W}_{N}^{+} {\to }^{{k}_{1}} {I}^{-} + {X}_{N}^{-} + {W}_{N}^{+} \qquad ( 3 d )$$
$${Y}^{+} + {H}^{+} {\to }^{{k}_{1}} {I}^{-} + {H}^{+} \qquad ( 4 a )$$
$${Y}^{-} + {H}^{-} {\to }^{{k}_{1}} {I}^{+} + {H}^{-} \qquad ( 4 b )$$
$${I}^{+} + {C}^{+} {\to }^{{k}_{1}} {I}^{+} + {C}^{+} + {Y}^{+} \qquad ( 4 c )$$
$${I}^{-} + {C}^{-} {\to }^{{k}_{1}} {I}^{-} + {C}^{-} + {Y}^{-} \qquad ( 4 d )$$
$${X}_{1}^{+} + {X}_{1}^{-} {\to }^{{k}_{2}} Φ \qquad ( 5 a )$$
$${W}_{1}^{+} + {W}_{1}^{-} {\to }^{{k}_{2}} Φ \qquad ( 5 b )$$
$${X}_{2}^{+} + {X}_{2}^{-} {\to }^{{k}_{2}} Φ \qquad ( 5 c )$$
$${W}_{2}^{+} + {W}_{2}^{-} {\to }^{{k}_{2}} Φ \qquad ( 5 d )$$
$$⋮$$
$${X}_{i}^{+} + {X}_{i}^{-} {\to }^{{k}_{2}} Φ \qquad ( 6 a )$$
$${W}_{i}^{+} + {W}_{i}^{-} {\to }^{{k}_{2}} Φ \qquad ( 6 b )$$
$${Y}^{+} + {Y}^{-} {\to }^{{k}_{2}} Φ \qquad ( 6 c )$$
$${C}^{+} + {C}^{-} {\to }^{{k}_{2}} Φ \qquad ( 6 d )$$
$${H}^{+} + {H}^{-} {\to }^{{k}_{2}} Φ \qquad ( 6 e )$$
$${I}^{+} + {I}^{-} {\to }^{{k}_{2}} Φ \qquad ( 6 f )$$
where the reaction expressions (1a) to (3d) belong to a first catalytic reaction module;
the reaction expressions (4a) and (4b) belong to a second catalytic reaction module; the reaction expressions (4c) and (4d) belong to the first catalytic reaction module; the reaction expressions (5a) to (6f) belong to an annihilation reaction module; k₁ and k₂ represent different reaction rates; and Φ represents a waste; Xᵢ(t)=[Xᵢ⁺]ₜ−[Xᵢ⁻]ₜ, i=1, 2, . . . , N, N represents the number of input items, [*]^{t} represents a concentration of a substance * at a time t, and Xᵢ represents an i-th input item; Xᵢ⁺ and Xᵢ⁻ respectively represent a positive part and a negative part of Xᵢ; Wᵢ(t)=[Wᵢ⁺]ₜ−[Wᵢ⁻]ₜ, Wᵢ represents an i-th weight corresponding to the i-th input item, Wᵢ⁺ and Wᵢ⁻ respectively represent a positive part and a negative part of Wᵢ; I⁺ and I⁻ respectively represent a positive part and a negative part of a substance I; H⁺ and H⁻ respectively represent a positive part and a negative part of a substance H; C⁺ and C⁻ respectively represent a positive part and a negative part of a substance C; supposing [H⁺]ₜ=[H⁻]ₜ=1 nanomoles per liter (nM), differential equations of concentrations varying over time of I⁺ and I⁻ are as follows:
$$\{ \begin{matrix}\frac{{d [ {I}^{+} ]}_{t}}{dt} = {k}_{1} ( {{[ {W}_{1}^{+} ]}_{t} [ {X}_{1}^{+} ]}_{t} + {{[ {W}_{2}^{+} ]}_{t} [ {X}_{2}^{+} ]}_{t} + … + {{[ {W}_{N}^{+} ]}_{t} [ {X}_{N}^{+} ]}_{t} \\ + {{[ {W}_{1}^{-} ]}_{t} [ {X}_{1}^{-} ]}_{t} + {{[ {W}_{2}^{-} ]}_{t} [ {X}_{2}^{-} ]}_{t} + … + {{[ {W}_{N}^{-} ]}_{t} [ {X}_{N}^{-} ]}_{t} \\ + {{[ {Y}^{-} ]}_{t} [ {H}^{-} ]}_{t} ) \\ \frac{{d [ {I}^{-} ]}_{t}}{dt} = {k}_{1} ( {{[ {W}_{1}^{+} ]}_{t} [ {X}_{1}^{-} ]}_{t} + {{[ {W}_{2}^{+} ]}_{t} [ {X}_{2}^{-} ]}_{t} + … + {{[ {W}_{N}^{+} ]}_{t} [ {X}_{N}^{-} ]}_{t} \\ + {{[ {W}_{1}^{-} ]}_{t} [ {X}_{1}^{+} ]}_{t} + {{[ {W}_{2}^{-} ]}_{t} [ {X}_{2}^{+} ]}_{t} + … + {{[ {W}_{N}^{-} ]}_{t} [ {X}_{N}^{+} ]}_{t} \\ + {{[ {Y}^{+} ]}_{t} [ {H}^{+} ]}_{t} )\end{matrix} \qquad ( 7 )$$
the differential equations (7) are simplified as follows:
$$\{ \begin{matrix}\frac{{d [ {I}^{+} ]}_{t}}{dt} = {k}_{1} ( {\sum }_{i = 1}^{N} ( {{[ {W}_{i}^{+} ]}_{t} [ {X}_{i}^{+} ]}_{t} + {{[ {W}_{i}^{-} ]}_{t} [ {X}_{i}^{-} ]}_{t} ) + {{[ {Y}^{-} ]}_{t} [ H ]}_{t} ) \\ \frac{{d [ {I}^{-} ]}_{t}}{dt} = {k}_{1} ( {\sum }_{i = 1}^{N} ( {{[ {W}_{t}^{+} ]}_{t} [ {X}_{i}^{-} ]}_{t} + {{[ {W}_{i}^{-} ]}_{t} [ {X}_{i}^{+} ]}_{t} ) + {{[ {Y}^{+} ]}_{t} [ {H}^{+} ]}_{t} )\end{matrix}$$
$$and$$
$$\frac{dI ( t )}{dt} = \frac{{d [ {I}^{+} ]}_{t}}{dt} - \frac{{d [ {I}^{-} ]}_{t}}{dt} = {k}_{1} [ {\sum }_{i = 1}^{N} ( {{[ {W}_{i}^{+} ]}_{t} [ {X}_{i}^{+} ]}_{t} + {{[ {W}_{i}^{-} ]}_{t} [ {X}_{i}^{-} ]}_{t} - {{[ {W}_{i}^{+} ]}_{t} [ {X}_{i}^{-} ]}_{t} - {{[ {W}_{i}^{-} ]}_{t} [ {X}_{i}^{+} ]}_{t} ) + {{[ {Y}^{-} ]}_{t} [ {H}^{-} ]}_{t} - {{[ {Y}^{+} ]}_{t} [ {H}^{+} ]}_{t} ]$$
supposing [H⁺]ₜ=[H⁻]ₜ≡1 nM, and
$$\frac{dI ( t )}{dt} = {k}_{1} ( {\sum }_{i = 1}^{N} {W}_{i} ( t ) {X}_{i} ( t ) - Y ( t ) ) ,$$
when a DNA hybridization reaction network reaches dynamic equilibrium, i.e.,
$$\frac{dI ( t )}{dt} = 0 ,$$
then:
$$Y = {\sum }_{i = 1}^{N} {W}_{i} {X}_{i}$$
where Y(t)=[Y⁺]ₜ−[Y⁻]ₜ, Y represents an output value of a system;
(2) reaction expressions of the algorithm part are as follows:
$${D}^{+} + {X}_{1}^{+} {\to }^{{k}_{3}} {D}^{+} + {X}_{1}^{+} + {W}_{1}^{+} \qquad ( 8 a )$$
$${D}^{-} + {X}_{1}^{-} {\to }^{{k}_{3}} {D}^{-} + {X}_{1}^{-} + {W}_{1}^{+} \qquad ( 8 b )$$
$${Y}^{+} + {X}_{1}^{-} {\to }^{{k}_{3}} {Y}^{+} + {X}_{1}^{-} + {W}_{1}^{+} \qquad ( 8 c )$$
$${Y}^{-} + {X}_{1}^{+} {\to }^{{k}_{3}} {D}^{-} + {X}_{1}^{+} + {W}_{1}^{+} \qquad ( 8 d )$$
$${D}^{+} + {X}_{1}^{-} {\to }^{{k}_{3}} {D}^{+} + {X}_{1}^{-} + {W}_{1}^{-} \qquad ( 8 e )$$
$${D}^{-} + {X}_{1}^{+} {\to }^{{k}_{3}} {D}^{-} + {X}_{1}^{+} + {W}_{1}^{-} \qquad ( 8 f )$$
$${Y}^{+} + {X}_{1}^{+} {\to }^{{k}_{3}} {Y}^{+} + {X}_{1}^{+} + {W}_{1}^{-} \qquad ( 8 g )$$
$${Y}^{-} + {X}_{1}^{-} {\to }^{{k}_{3}} {D}^{-} + {X}_{1}^{-} + {W}_{1}^{-} \qquad ( 8 h )$$
$${D}^{+} + {X}_{2}^{+} {\to }^{{k}_{3}} {D}^{+} + {X}_{2}^{+} + {W}_{2}^{+} \qquad ( 9 a )$$
$${D}^{-} + {X}_{2}^{-} {\to }^{{k}_{3}} {D}^{-} + {X}_{2}^{-} + {W}_{2}^{+} \qquad ( 9 b )$$
$${Y}^{+} + {X}_{2}^{+} {\to }^{{k}_{3}} {Y}^{+} + {X}_{2}^{-} + {W}_{2}^{+} \qquad ( 9 c )$$
$${Y}^{-} + {X}_{2}^{+} {\to }^{{k}_{3}} {D}^{-} + {X}_{2}^{+} + {W}_{2}^{+} \qquad ( 9 d )$$
$${D}^{+} + {X}_{2}^{-} {\to }^{{k}_{3}} {D}^{+} + {X}_{2}^{-} + {W}_{2}^{-} \qquad ( 9 e )$$
$${D}^{-} + {X}_{2}^{+} {\to }^{{k}_{3}} {D}^{-} + {X}_{2}^{+} + {W}_{2}^{-} \qquad ( 9 f )$$
$${Y}^{+} + {X}_{2}^{+} {\to }^{{k}_{3}} {Y}^{+} + {X}_{2}^{+} + {W}_{2}^{-} \qquad ( 9 g )$$
$${Y}^{-} + {X}_{2}^{-} {\to }^{{k}_{3}} {D}^{-} + {X}_{2}^{-} + {W}_{2}^{-} \qquad ( 9 h )$$
$$⋮$$
$${D}^{+} + {X}_{N}^{+} {\to }^{{k}_{3}} {D}^{+} + {X}_{N}^{+} + {W}_{N}^{+} \qquad ( 10 a )$$
$${D}^{-} + {X}_{N}^{-} {\to }^{{k}_{3}} {D}^{-} + {X}_{N}^{-} + {W}_{N}^{+} \qquad ( 10 b )$$
$${Y}^{+} + {X}_{N}^{-} {\to }^{{k}_{3}} {Y}^{+} + {X}_{N}^{-} + {W}_{N}^{+} \qquad ( 10 c )$$
$${Y}^{-} + {X}_{N}^{+} {\to }^{{k}_{3}} {D}^{-} + {X}_{N}^{+} + {W}_{N}^{+} \qquad ( 10 d )$$
$${D}^{+} + {X}_{N}^{-} {\to }^{{k}_{3}} {D}^{+} + {X}_{N}^{-} + {W}_{N}^{-} \qquad ( 10 e )$$
$${D}^{-} + {X}_{N}^{+} {\to }^{{k}_{3}} {D}^{-} + {X}_{N}^{+} + {W}_{N}^{-} \qquad ( 10 f )$$
$${Y}^{+} + {X}_{N}^{+} {\to }^{{k}_{3}} {Y}^{+} + {X}_{N}^{+} + {W}_{N}^{-} \qquad ( 10 g )$$
$${Y}^{-} + {X}_{N}^{+} {\to }^{{k}_{3}} {D}^{-} + {X}_{N}^{-} + {W}_{N}^{-} \qquad ( 10 h )$$
where the reaction expressions (8) to (10) belong to the first catalytic reaction module; D⁺ and D⁻ respectively represent a positive part and a negative part of D; and k₃ represents a reaction rate which is different from k₁ and k₂;
differential equations of concentrations varying over time of Wᵢ⁺ and Wᵢ⁻ are as follows:
$$\{ \begin{matrix}\frac{d [ {W}_{1}^{+} ]}{dt} = {k}_{3} ( {{[ {D}^{+} ]}_{t} [ {X}_{1}^{+} ]}_{t} + {{[ {D}^{-} ]}_{t} [ {X}_{1}^{-} ]}_{t} + {{[ {Y}^{+} ]}_{t} [ {X}_{1}^{-} ]}_{t} + {{[ {Y}^{-} ]}_{t} [ {X}_{1}^{+} ]}_{t} ) \\ \frac{d [ {W}_{1}^{-} ]}{dt} = {k}_{3} ( {{[ {D}^{+} ]}_{t} [ {X}_{1}^{-} ]}_{t} + {{[ {D}^{-} ]}_{t} [ {X}_{1}^{+} ]}_{t} + {{[ {Y}^{+} ]}_{t} [ {X}_{1}^{+} ]}_{t} + {{[ {Y}^{-} ]}_{t} [ {X}_{1}^{-} ]}_{t} )\end{matrix} \qquad ( 11 )$$
$$\{ \begin{matrix}\frac{d [ {W}_{2}^{+} ]}{dt} = {k}_{3} ( {{[ {D}^{+} ]}_{t} [ {X}_{2}^{+} ]}_{t} + {{[ {D}^{-} ]}_{t} [ {X}_{2}^{-} ]}_{t} + {{[ {Y}^{+} ]}_{t} [ {X}_{2}^{-} ]}_{t} + {{[ {Y}^{-} ]}_{t} [ {X}_{2}^{+} ]}_{t} ) \\ \frac{d [ {W}_{2}^{-} ]}{dt} = {k}_{3} ( {{[ {D}^{+} ]}_{t} [ {X}_{2}^{-} ]}_{t} + {{[ {D}^{-} ]}_{t} [ {X}_{2}^{+} ]}_{t} + {{[ {Y}^{+} ]}_{t} [ {X}_{2}^{+} ]}_{t} + {{[ {Y}^{-} ]}_{t} [ {X}_{2}^{-} ]}_{t} )\end{matrix} \qquad ( 12 )$$
$$\{ \begin{matrix}\frac{d [ {W}_{N}^{+} ]}{dt} = {k}_{3} ( {{[ {D}^{+} ]}_{t} [ {X}_{N}^{+} ]}_{t} + {{[ {D}^{-} ]}_{t} [ {X}_{N}^{-} ]}_{t} + {{[ {Y}^{+} ]}_{t} [ {X}_{N}^{-} ]}_{t} + {{[ {Y}^{-} ]}_{t} [ {X}_{N}^{+} ]}_{t} ) \\ \frac{d [ {W}_{N}^{-} ]}{dt} = {k}_{3} ( {{[ {D}^{+} ]}_{t} [ {X}_{N}^{-} ]}_{t} + {{[ {D}^{-} ]}_{t} [ {X}_{N}^{+} ]}_{t} + {{[ {Y}^{+} ]}_{t} [ {X}_{N}^{+} ]}_{t} + {{[ {Y}^{-} ]}_{t} [ {X}_{N}^{-} ]}_{t} )\end{matrix} \qquad ( 13 )$$
wherein D(t)=[D⁺]ₜ−[D⁻]ₜ, D represents an expectation value;
based on the differential equations (11) to (13), expressions are obtained as follows:
$$\begin{matrix}\frac{{dW}_{1} ( t )}{dt} = \frac{{d [ {W}_{1}^{+} ]}_{t}}{dt} - \frac{{d [ {W}_{1} ]}_{t}}{dt} \\ = {k}_{3} ( {{[ {D}^{+} ]}_{t} [ {X}_{1}^{+} ]}_{t} + {{[ {D}^{-} ]}_{t} [ {X}_{1}^{-} ]}_{t} - {{[ {D}^{+} ]}_{t} [ {X}_{1}^{-} ]}_{t} - {{[ {D}^{-} ]}_{t} [ {X}_{1}^{+} ]}_{t} ) \\ + {k}_{2} ( {{[ {Y}^{+} ]}_{t} [ {X}_{1}^{-} ]}_{t} + {{[ {Y}^{-} ]}_{t} [ {X}_{1}^{+} ]}_{t} - {{[ {Y}^{+} ]}_{t} [ {X}_{1}^{+} ]}_{t} + {{[ {Y}^{-} ]}_{t} [ {X}_{1}^{-} ]}_{t} ) \\ = {k}_{3} [ ( {[ {D}^{+} ]}_{t} - {[ {D}^{-} ]}_{t} ) - ( {[ {Y}^{+} ]}_{t} - {[ {Y}^{-} ]}_{t} ) ] \times ( {[ {X}_{1}^{+} ]}_{t} - {[ {X}_{1}^{-} ]}_{t} ) \\ = {{k}_{3} ( {[ D ]}_{t} - {[ Y ]}_{t} ) [ {X}_{1} ]}_{t}\end{matrix}$$
$$\begin{matrix}\frac{{dW}_{2} ( t )}{dt} = \frac{{d [ {W}_{2}^{+} ]}_{t}}{dt} - \frac{{d [ {W}_{2} ]}_{t}}{dt} \\ = {k}_{3} ( {{[ {D}^{+} ]}_{t} [ {X}_{2}^{+} ]}_{t} + {{[ {D}^{-} ]}_{t} [ {X}_{2}^{-} ]}_{t} - {{[ {D}^{+} ]}_{t} [ {X}_{2}^{-} ]}_{t} - {{[ {D}^{-} ]}_{t} [ {X}_{2}^{+} ]}_{t} ) \\ + {k}_{2} ( {{[ {Y}^{+} ]}_{t} [ {X}_{2}^{-} ]}_{t} + {{[ {Y}^{-} ]}_{t} [ {X}_{2}^{+} ]}_{t} - {{[ {Y}^{+} ]}_{t} [ {X}_{2}^{+} ]}_{t} + {{[ {Y}^{-} ]}_{t} [ {X}_{2}^{-} ]}_{t} ) \\ = {k}_{3} [ ( {[ {D}^{+} ]}_{t} - {[ {D}^{-} ]}_{t} ) - ( {[ {Y}^{+} ]}_{t} - {[ {Y}^{-} ]}_{t} ) ] \times ( {[ {X}_{2}^{+} ]}_{t} - {[ {X}_{2}^{-} ]}_{t} ) \\ = {{k}_{3} ( {[ D ]}_{t} - {[ Y ]}_{t} ) [ {X}_{2} ]}_{t}\end{matrix}$$
$$⋮$$
$$\begin{matrix}\frac{{dW}_{N} ( t )}{dt} = \frac{{d [ {W}_{N}^{+} ]}_{t}}{dt} - \frac{{d [ {W}_{N}^{-} ]}_{t}}{dt} \\ = {k}_{3} ( {{[ {D}^{+} ]}_{t} [ {X}_{N}^{+} ]}_{t} + {{[ {D}^{-} ]}_{t} [ {X}_{N}^{-} ]}_{t} - {{[ {D}^{+} ]}_{t} [ {X}_{N}^{-} ]}_{t} - {{[ {D}^{-} ]}_{t} [ {X}_{N}^{*} ]}_{t} ) \\ + {k}_{2} ( {{[ {Y}^{+} ]}_{t} [ {X}_{N}^{-} ]}_{t} + {{[ {Y}^{-} ]}_{t} [ {X}_{N}^{*} ]}_{t} \\ = {k}_{3} [ ( {[ {D}^{+} ]}_{t} - {[ {D}^{-} ]}_{t} ) - ( {[ {Y}^{+} ]}_{t} - {[ {Y}^{-} ]}_{f} ) ] \times ( {[ {X}_{N}^{+} ]}_{t} - {[ {X}_{N}^{-} ]}_{t} ) \\ = {{k}_{3} ( {[ D ]}_{t} - {[ Y ]}_{t} ) [ {X}_{N} ]}_{t} ;\end{matrix}$$
(3) reaction expressions of the testing part are as follows:
$${X}_{1}^{+} + {\hat{W}}_{1}^{+} {\to }^{{k}_{1}} {\hat{I}}^{+} + {X}_{1}^{+} + {\hat{W}}_{1}^{+} \qquad ( 14 a )$$
$${X}_{1}^{-} + {\hat{W}}_{1}^{-} {\to }^{{k}_{1}} {\hat{I}}^{+} + {X}_{1}^{-} + {\hat{W}}_{1}^{+} \qquad ( 14 b )$$
$${X}_{1}^{+} + {\hat{W}}_{1}^{-} {\to }^{{k}_{1}} {\hat{I}}^{-} + {X}_{1}^{+} + {\hat{W}}_{1}^{-} \qquad ( 14 c )$$
$${X}_{1}^{-} + {\hat{W}}_{1}^{+} {\to }^{{k}_{1}} {\hat{I}}^{-} + {X}_{1}^{-} + {\hat{W}}_{1}^{+} \qquad ( 14 d )$$
$${X}_{2}^{+} + {\hat{W}}_{2}^{+} {\to }^{{k}_{1}} {\hat{I}}^{+} + {X}_{2}^{+} + {\hat{W}}_{2}^{+} \qquad ( 15 a )$$
$${X}_{2}^{+} + {\hat{W}}_{2}^{+} {\to }^{{k}_{1}} {\hat{I}}^{+} + {X}_{2}^{+} + {\hat{W}}_{2}^{+} \qquad ( 15 b )$$
$${X}_{2}^{+} + {\hat{W}}_{2}^{-} {\to }^{{k}_{1}} {\hat{I}}^{-} + {X}_{2}^{+} + {\hat{W}}_{2}^{-} \qquad ( 15 c )$$
$${X}_{2}^{-} + {\hat{W}}_{2}^{+} {\to }^{{k}_{1}} {\hat{I}}^{-} + {X}_{2}^{-} + {\hat{W}}_{2}^{+} \qquad ( 15 d )$$
$$⋮$$
$${X}_{N}^{+} + {\hat{W}}_{N}^{+} {\to }^{{k}_{1}} {\hat{I}}^{+} + {X}_{N}^{+} + {\hat{W}}_{N}^{+} \qquad ( 16 a )$$
$${X}_{N}^{-} + {\hat{W}}_{N}^{-} {\to }^{{k}_{1}} {\hat{I}}^{+} + {X}_{N}^{-} + {\hat{W}}_{N}^{-} \qquad ( 16 b )$$
$${X}_{N}^{+} + {\hat{W}}_{N}^{-} {\to }^{{k}_{1}} {\hat{I}}^{-} + {X}_{N}^{+} + {\hat{W}}_{N}^{-} \qquad ( 16 c )$$
$${X}_{N}^{-} + {\hat{W}}_{N}^{+} {\to }^{{k}_{1}} {\hat{I}}^{-} + {X}_{N}^{-} + {\hat{W}}_{N}^{+} \qquad ( 16 d )$$
$${\hat{Y}}^{-} + {\hat{H}}^{-} {\to }^{{k}_{1}} {\hat{I}}^{-} + {\hat{H}}^{-} \qquad ( 17 a )$$
$${\hat{Y}}^{-} + {\hat{H}}^{-} {\to }^{{k}_{1}} {\hat{I}}^{-} + {\hat{H}}^{+} \qquad ( 17 b )$$
$${\hat{I}}^{+} + {\hat{C}}^{+} {\to }^{{k}_{1}} {\hat{I}}^{+} + {\hat{C}}^{+} + {\hat{Y}}^{+} \qquad ( 17 c )$$
$${\hat{I}}^{-} + {\hat{C}}^{-} {\to }^{{k}_{1}} {\hat{I}}^{-} + {\hat{C}}^{-} + {\hat{Y}}^{+} \qquad ( 17 d )$$
$${X}_{i}^{+} + {X}_{i}^{-} {\to }^{{k}_{2}} Φ \qquad ( 18 a )$$
$${\hat{W}}_{i}^{+} + {\hat{W}}_{i}^{-} {\to }^{{k}_{2}} Φ \qquad ( 18 b )$$
$${\hat{Y}}^{+} + {\hat{Y}}^{-} {\to }^{{k}_{2}} Φ \qquad ( 18 c )$$
$${\hat{C}}^{+} + {\hat{C}}^{-} {\to }^{{k}_{2}} Φ \qquad ( 18 d )$$
$${\hat{H}}^{+} + {\hat{H}}^{-} {\to }^{{k}_{2}} Φ \qquad ( 18 e )$$
$${\hat{I}}^{+} + {\hat{I}}^{-} {\to }^{{k}_{2}} Φ \qquad ( 18 f )$$
where the reaction expressions (14d) to (16d) belong to the first catalytic reaction module;
the reaction expressions (17a) and (17b) belong to the second catalytic reaction module; the reaction expressions (17c) and (17d) belong to the first catalytic reaction module; and the reaction expressions (18a) to (18f) belong to the annihilation reaction module; Ŵᵢ represents a weight obtained after a plurality of rounds of learning; Ŵᵢ⁺ and \tilde{W}ᵢ⁻ respectively represent a positive part and a negative part of Ŵᵢ; Ŷ represents an output of the testing part; Ŷ⁺ and Ŷ⁻ respectively represent a positive part and a negative part of Ŷ; Î, Ĥ and Ĉ represent substances involved in a chemical reaction in the testing part; Ĥ⁺ and Ĥ⁻ respectively represent a positive part and a negative part of Ĥ; Ĉ⁺ and Ĉ⁻ respectively represent a positive part and a negative part of Ĉ; Î⁺ and Î⁻ respectively represent a positive part and a negative part of Î;
the linear machine learning method comprises following steps S1-S3:
S1, normalizing training data; wherein training of machine learning comprises a plurality of rounds of training; each of the plurality of rounds of training comprises: K sets of training data, each set of training data comprises N data, and the machine learning has N inputs and i=1, 2, 3, . . . , N;
shuffling the K sets of training data to obtain another round of training data, and normalizing the another round of training data as follows:
$$\{ \begin{matrix}{\hat{x}}_{i} ( k , l ) = ρ {x}_{i} ( k , l ) / η \\ {\hat{d}}_{l} ( k ) = ρ {\tilde{d}}_{l} ( k ) / η\end{matrix} \qquad ( 19 )$$
where:
$$\{ \begin{matrix}{ε}_{i} = max ( {Θ}_{i} ) - min ( {Θ}_{i} ) \\ η = max ( [ {ε}_{1} , {ε}_{2} , … {ε}_{N} ] ) \\ {\tilde{y}}_{l} ( k ) = {\sum }_{i = 1}^{N} {w}_{i} {x}_{i} ( l , k )\end{matrix} ;$$
xᵢ(k,l) represents an i-th data of a k-th set of an l-th round of training, k=1, 2, . . . , K, l=1, 2, . . . , ∧, ρ represents a positive adjustment parameter, Θᵢ=[xᵢ(1), xᵢ(2), . . . , xᵢ(K)] and \hat{d}ₗ (k) meeting \hat{x}ᵢ(k,l)=[Xᵢ⁺]₀−[Xᵢ⁻]₀, and \hat{d}ₗ(k)=[D⁺]₀−[D⁻]₀;
S2, assessing the training of the machine learning;
defining a relative error eₗ(k) in the l-th round of training as follows:
$${e}_{l} ( k ) = \frac{{\tilde{y}}_{l} ( k ) - {\hat{d}}_{l} ( k )}{{\hat{d}}_{l} ( k )} \qquad ( 20 )$$
where:
$${\hat{y}}_{n} ( k ) = {\sum }_{i = 1}^{N} {\hat{W}}_{i} {χ}_{i} ( k ) ;$$
assessing a result of the round of training and defining an average relative error as follows:
$${\overline{e}}_{l} = \frac{1}{K} {\sum }_{k = 1}^{K} \vert {e}_{l} ( k ) \vert \qquad ( 21 )$$
performing the plurality of rounds of trainings, and stopping training after the average relative error reaches a target value;
S3, assessing and testing of the machine learning;
the testing of machine learning comprising a plurality of rounds of testing; one of the plurality of rounds of testing comprising P sets of test data; shuffling the P sets of test data to obtain another round of test data, and normalizing the another round of test data as follows:
$$\{ \begin{matrix}{\hat{x}}_{i}^{′} ( p , g ) = {ρβ}_{i} ( p , g ) / θ \\ {\hat{d}}_{g}^{′} ( p ) = ρ {\tilde{d}}_{g}^{′} ( p ) / θ\end{matrix}$$
βᵢ(p,g) represents an i-th data of a p-th set of a g-th round of testing, p=1, 2, . . . , P, g=1,2, . . . ,G; θᵢ=max(Φᵢ)−min(Φᵢ), θ=max ([θ₁, θ₂, . . . θ_{N}]), and \tilde{d}′_{g}(p)=Σᵢ₌₁^{N}wᵢxᵢ(g,p);
in the g-th round of testing, defining a relative error e′_{g} (p) as follows:
$${e}_{g}^{′} ( p ) = \frac{{\tilde{y}}_{g}^{′} ( p ) - {\hat{d}}_{g}^{′} ( p )}{{\hat{d}}_{l}^{′} ( p )} ,$$
where:
$${\hat{y}}_{g} ( p ) = {\sum }_{i = 1}^{N} {\hat{W}}_{i} {\hat{x}}_{i}^{′} ( k ) ;$$
assessing a result of the round of training, and defining an average relative error in the testing as follows:
$${\overline{e}}_{g}^{′} = \frac{1}{P} {\sum }_{p = 1}^{P} \vert {e}_{g} ( k ) \vert .$$

2. The linear machine learning method as claimed in claim 1, wherein a reaction expression of the first catalytic reaction module is:
$${X}_{i}^{+} + {W}_{i}^{+} {\to }^{{k}_{i}} {I}^{+} + {X}_{i}^{+} + {W}_{i}^{+} ,$$
which is obtained by following DNA strand displacement reactions:
$$\{ \begin{matrix}{W}_{i}^{+} + {Ap}_{i}^{+} {⇄}_{{q}_{m}}^{{q}_{i}} {Ad}_{i}^{+} + {Aq}_{i}^{+} \\ {Ad}_{i}^{+} + {X}_{i}^{+} {\to }^{{q}_{m}} {Ac}_{i}^{+} + waste \\ {Ac}_{i}^{+} + {Ab}_{i}^{+} {\to }^{{q}_{m}} {I}^{+} + {X}_{i}^{+} + {W}_{i}^{+} + waste\end{matrix} \qquad ( 22 )$$
wherein I⁺ is catalyzed, Apᵢ⁺, Aqᵢ⁺ and Abᵢ⁺ are auxiliary DNA strands and initial concentrations of the auxiliary DNA strands are Cₘ, meeting Cₘ≥[Xᵢ⁺]₀,[Wᵢ⁺]₀,[I⁺]₀; reaction rates qᵢ and kᵢ meet qᵢ≤qₘ, kᵢ=qᵢ; and qₘ represents a maximum reaction rate; Adᵢ⁺, Abᵢ⁺ and Acᵢ⁺ represent generated DNA strands in the first catalytic reaction module;
a reaction expression of the second catalytic reaction module is:
$${Y}^{+} + {H}^{+} {\to }^{{k}_{i}} {I}^{-} + {H}^{+} ,$$
which is obtained by following DNA strand displacement reactions:
$$\{ \begin{matrix}{Y}^{+} + {Am}^{+} {⇄}_{{q}_{m}}^{{q}_{i}} {An}^{+} + {Ae}^{+} \\ {Ae}^{+} + {H}^{+} {\to }^{{q}_{m}} {As}^{+} + waste \\ {As}^{+} + {Ah}^{+} {\to }^{{q}_{m}} {H}^{+} + {I}^{-} + waste\end{matrix} \qquad ( 23 )$$
wherein I⁻ is catalyzed, Am⁺, An⁺, Ah⁺ and H⁺ are auxiliary DNA strands and initial concentrations of the auxiliary DNA strands Am⁺, An⁺ and Ah⁺ are respectively set [Am⁺]₀=[An⁺]₀=[Ah⁺]₀=Cₘ, meeting Cₘ□[Y⁺]₀,[I⁻]₀; an initial concentration of H⁺ is 1 nM; and the reaction rate qᵢ meets qᵢ≤qₘ, kᵢ=qᵢ; Ae⁺ and As⁺ represent generated DNA strands in the second catalyzation reaction module;
a reaction expression of the annihilation reaction module is:
$${W}_{i}^{+} + {W}_{i}^{-} {\to }^{{k}_{i}} Φ ,$$
which is obtained by following DNA strand displacement reactions:
$$\{ \begin{matrix}{W}_{i}^{+} + {Wa}_{i}^{+} {⇄}_{{q}_{m}}^{{q}_{i}} {Wb}_{i}^{+} + {Wt}_{i}^{+} \\ {Wt}_{i}^{+} + {W}_{i}^{-} {\to }^{{q}_{m}} Φ\end{matrix} \qquad ( 24 )$$
wherein Wᵢ⁺ and Wᵢ⁻ are annihilated, Waᵢ⁺ and Wbᵢ⁻ r are auxiliary DNA strands and initial concentrations of the auxiliary DNA strands are Cₘ, meeting Cₘ□[Wᵢ⁺]₀,[Wᵢ⁻]₀; and the reaction rate qᵢ meets qᵢ≤qₘ, kᵢ=qᵢ.

3. The linear machine learning method as claimed in claim 1, wherein the first catalytic reaction module, the second catalytic reaction module and the annihilation reaction module in the training part, the algorithm part and the testing part belong to a same type of reaction module but are not a same reaction module.
